(19) United States
(12) Patent Application Publication
(10) Pub. No.: US 2025/0339454 A1
(43) Pub. Date: Nov. 6, 2025

(54) COMPOSITIONS AND METHODS FOR THE PREVENTION OF LOSS OF ORGAN FUNCTION ASSOCIATED WITH CHRONIC ORGAN DISEASE

(71) Applicant: Revelation Biosciences, Inc., San Diego, CA (US)

(72) Inventors: James M. Rolke, Tucson, AZ (US); Robin L. Marsden, EI Cajon, CA (US); Kristine Storey, San Diego, CA (US); Sima Patel, San Diego, CA (US)

(21) Appl. No.: 18/864,754

(22) PCT Filed: May 12, 2023
(86) PCT No.: PCT/US2023/022019

§ 371 Date: Nov. 11, 2024

(51) Int. Cl. A61K 31/7028; A61K 9/08; A61K 9/107; A61K 9/14; A61K 47/26; A61K 47/38; A61K 47/40; A61P 13/12

(52) U.S. Cl. CPC A61K 31/7028; A61K 9/08; A61K 9/107; A61K 9/145; A61K 9/146; A61K 47/26; A61K 47/38; A61K 47/40; A61P 13/12

(57) ABSTRACT

The present disclosure provides methods for treating or preventing loss of organ function due to chronic organ or tissue diseases by administering to a subject an effective amount of a TLR4 agonist, such as an MPLA-like compound. More particularly, the present disclosure provides methods for preventing loss of kidney function due to chronic kidney disease, methods for preventing loss of organ function due to acute stress, and methods for preventing loss of kidney function due to acute stress by administering to a subject an effective amount of a TLR4 agonist. Also provided are pharmaceutical compositions comprising a TLR4 agonist that are useful for carrying out the aforesaid methods.

### CROSS-REFERENCE TO RELATED APPLICATIONS

This application claims the benefit of priority to U.S. Provisional Application No. 63/341,209 filed on May 12, 2022, the contents of which are hereby incorporated by reference in their entirety.

### BACKGROUND

Organ damage, due to chronic disease, is a pervasive problem in the United States and world-wide. Organ disease (due to chronic inflammation and subsequent fibrosis, for example) is progressive and ultimately results in loss of function of the organ. Examples of chronic organ disease include chronic kidney disease (CKD) through end-stage renal disease, liver diseases such as non-alcoholic steatohepatitis (NASH), osteoarthritis, rheumatoid arthritis, pulmonary fibrotic disease, heart disease, pancreatitis, cancer, and irritable bowel syndrome.

In addition, acute organ stress may lead to reduced functionality and eventual decline, contributing to a chronic organ disease. One well known example of this is acute kidney toxicity (the stress) associated with platinum-based chemotherapy, which often leads to the development of chronic kidney disease.

There are currently no effective therapies for treating chronic organ disease or therapies for preventing loss of function due to acute organ dysfunction, outside of steroidal treatment or organ transplantation.

Many acute and chronic disease states are caused by perpetual inflammation via the activity of the innate immune system in response to a specific stressor. The innate immune system is our first line of defense against stress from such things as toxic chemicals, injury, invading pathogens such as bacteria, viruses and fungi, and underlying disease. Examples of these chronic disease states include the propagation of chronic kidney disease by diabetes or the propagation of NASH due to inflammation as a result of diabetes or obesity. The innate immune system defends against stress by releasing cytokines and chemokines in response to stimulation of various families of receptors including pattern recognition receptors (PRRs) which recognize a range of pathogenic molecules, such as pathogen associated molecular pattern receptors (PAMPs) and damage associated molecular pattern receptors (DAMPs). In a healthy individual, cytokines and chemokines are proteins that direct different cellular activities to combat and resolve stress induced damage. Cytokine dysregulation, or an imbalance in the normal cytokine response, may not only initiate and cause chronic inflammation, but may also contribute to existing chronic inflammation leading to organ disease.

Toll-like receptors (TLRs) are a family of receptors that serve a vital role in initiating the innate immune response by recognizing different molecular patterns associated with pathogens such as bacteria and viruses as well as proteins that may cause cellular and tissue damage.

Stimulation of toll-like receptor 4 (TLR4), can activate one of two pathways: 1) the Myeloid differentiation primary response 88 (MyD88) pathway, which leads to the production of pro-inflammatory cytokines, and 2) the TIR-domain-clustering adapter-inducing interferon-β (TRIF) pathway which leads to the production of anti-inflammatory protective cytokines and type I interferons, such as interferon-β (FIG. 1).

Formulation of MPLA like compounds is difficult due the hydrophobic nature of the molecule and the potential need for the formation of a stable micelle to impart improved biologic activity. While multiple formulations have been described including oil-in-water emulsions, suspensions, nanoparticulate suspensions, liposomal formulations, and aqueous formulations with a cosurfactant, these all suffer from multiple drawbacks including injection site pain, injection site reaction, lack of bioavailability, inability to be given orally, inability to be given parenterally, lack of stability, and/or lack of utility due to requirements for specialized equipment at the time of use.

Disclosed herein are compositions of MPLA like compounds and methods that are useful for slowing or stopping the progression of organ disease or tissue damage as a result of chronic inflammation and chronic fibrosis. In addition, compositions and methods for preventing organ disfunction due to an acute stress are disclosed.

### SUMMARY OF THE INVENTION

Chronic disease of an organ, due to chronic inflammation and subsequent fibrosis, follows a pattern of perpetual and ongoing destruction of living functional cells and subsequent replacement by the non-functional protein, collagen, resulting in fibrosis (scar tissue) (Wilson). The establishment of fibrosis and subsequent death of the organ is driven by ongoing inflammatory processes associated with the innate immune response. Redirection of the innate immune response from a pro-inflammatory state to an anti-inflammatory (or non-inflammatory, protective) state will rebalance the innate immune response to slow down or halt the progressive destruction and scarring of organ tissue, allowing the healing process to take place.

The current invention contemplates using MPLA like compounds as treatment to redirect the innate immune response from a pro-inflammatory state to an anti-inflammatory state, to restore a more normal level of function.

In some embodiments, the present invention provides a method for treating or preventing loss of organ function due to chronic organ diseases by administering to a subject in need thereof an effective amount of a toll-like receptor 4 (TLR4) agonist.

In some preferred embodiments, the chronic organ disease is selected from nonalcoholic fatty liver, nonalcoholic steatohepatitis, osteoarthritis, rheumatoid arthritis, irritable bowel syndrome, pulmonary fibrotic disease, heart disease, and any combination thereof.

In some embodiments, the method prevents loss of organ function due to chronic organ disease, while in other embodiments, the method treats loss of organ function due to chronic organ disease.

In other embodiments, the invention provides a method for treating or preventing loss of kidney function due to chronic kidney disease by administering to a subject in need thereof an effective amount of a toll-like receptor 4 (TLR4) agonist.

In some embodiments, the method prevents loss of loss of kidney function due to chronic kidney disease, while in other embodiments, the method treats loss of loss of kidney function due to chronic kidney disease.

The present invention further provides a method for treating or preventing loss of organ function due to acute stress by administering to a subject in need thereof an effective amount of a TLR4 agonist.

In certain embodiments, the organ is a kidney. In this and other embodiments, the acute stress is due to one or more of toxicity from a medication, toxicity from chemotherapy, ischemia, trauma, cancer, or infection. In some embodiments, the loss of organ function is due chronic inflammation or fibrosis.

In some embodiments, the method prevents loss of organ function due to acute stress. In this and other embodiments, the method treats loss of organ function due to acute stress.

In certain embodiments, the TLR4 agonist is an MPLA-like compound, such as phosphorylated hexaacyl disaccharide (PHAD), 3-deacyl phosphorylated hexaacyl disaccharide (3D-PHAD), 3D-(6-acyl) phosphorylated hexaacyl disaccharide (3D(6-acyl) PHAD) or pharmaceutically acceptable salts thereof, and more preferably PHAD or a pharmaceutically acceptable salt thereof.

In some embodiments, the MPLA-like compound is administered as an aqueous solution, preferably parenterally as an aqueous solution. In other embodiments, the MPLA-like compound is delivered orally as a tablet.

In some embodiments, the TLR4 agonist selectively stimulates the TIR-domain-clustering adapter-inducing interferon-β (TRIF) pathway.

In some embodiments, the TLR4 agonist reduces circulating TGF-β in the subject. In this and other embodiments, the TLR4 agonist increases circulating interleukin-10 (IL-10), circulating hepcidin and/or circulating neutrophil gelatinase-associated lipocalin (NGAL) in the subject.

The present invention further provides a pharmaceutical composition for treating or preventing loss of organ function in a subject in need thereof comprising a colloidal formulation of a monophosphoryl lipid A (MPLA)-like compound or a pharmaceutically acceptable salt thereof. In certain embodiments, the MPLA-like compound is phosphorylated hexaacyl disaccharide (PHAD), PHAD-504, 3D-(6-acyl)-PHAD, 3D-PHAD, or any combination thereof, or a pharmaceutically acceptable salt thereof. In certain preferred embodiments, the MPLA-like compound is PHAD or a pharmaceutically acceptable salt thereof.

In some embodiments, the pharmaceutical composition is an aqueous composition. In other embodiments, the pharmaceutical composition is a dry powder.

In some embodiments, the pharmaceutical composition has an MPLA concentration of about 1 μg/mL to about 10,000 μg/mL

In some embodiments, the composition further comprises a stabilizer, preferably stabilizer is trehalose.

In some embodiments, the composition comprises micelles having an average diameter or length of about 1 nm to about 1000 nm.

In some embodiments, the composition comprises a bulking agent selected from one or more of the following: mannitol, trehalose, chitosan, HP-B-Cyclodextrin, hydroxypropylmethylcellulose (HPMC), dextran, pea starch, and sucrose.

### BRIEF DESCRIPTION OF THE DRAWINGS

FIG. 1: A. Stimulation of toll-like receptor 4 (TLR4), can activate one of two pathways: 1) the Myeloid differentiation primary response 88 (MyD88) pathway which leads to the production of pro-inflammatory cytokines, and 2) the TIR-domain-clustering adapter-inducing interferon-b (TRIF) pathway which leads to the production of anti-inflammatory protective cytokines; B. Monophosphorolipid A (MPLA) and MPLA-like compounds can preferentially stimulate TLR4 to activate the TRIF pathway leading to the production of protective cytokines

FIG. 2: IP-10 upregulation as a function of dose using representative formulated PHAD. Multiple preparations of PHAD were assessed in an in vitro cell based activity assay using mouse macrophages of the J774 cell line to demonstrate stimulation of TLR4 in response to PHAD as measured by upregulation of IP-10.

FIG. 3: Dose-dependent reduction in fibrosis (collagen fraction volume) as a function of total collagen deposition measured via PicoSirius Red in a rat UUO model of acute kidney injury and chronic kidney disease, in response to daily treatment with PHAD (REVTx-300), an MPLA-like compound.

FIG. 4: Concentration of TGF-β in Rat Serum at Day 7 Post UUO Procedure in a rat model of acute kidney injury and chronic kidney disease in response to daily treatment with PHAD (REVTx-300) an MPLA-like compound.

FIG. 5: Concentration of NGAL in Rat Serum at Day 7 Post UUO Procedure in a rat model of acute kidney injury and chronic kidney disease in response to daily treatment with PHAD (REVTx-300) an MPLA-like compound.

FIG. 6: Concentration of Hepcidin in Rat Serum at Day 7 Post UUO Procedure in a rat model of acute kidney injury and chronic kidney disease in response to daily treatment with PHAD (REVTx-300) an MPLA-like compound.

FIG. 7: Concentration of Hemeoxygenase-1 in Rat Serum at Day 7 Post UUO Procedure in a rat model of acute kidney injury and chronic kidney disease in response to daily treatment with PHAD (REVTx-300) an MPLA-like compound.

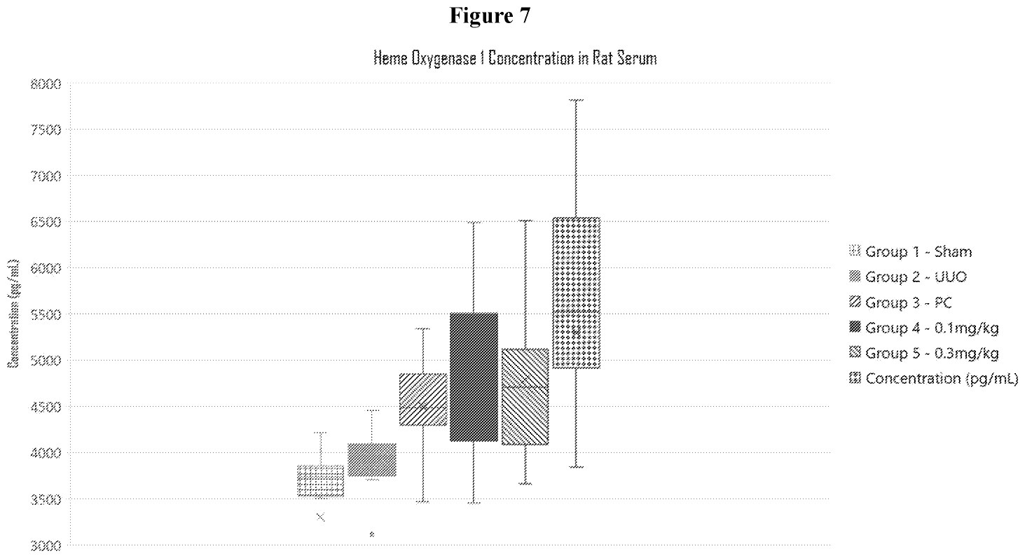

FIG. 8: Concentration of IL-10 in Rat Serum at Day 7 Post UUO Procedure in a rat model of acute kidney injury and chronic kidney disease in response to daily treatment with PHAD (REVTx-300) an MPLA-like compound.

FIG. 9: Concentration of IL-1B in Rat Serum at Day 7 Post UUO Procedure in a rat model of acute kidney injury and chronic kidney disease in response to daily treatment with PHAD (REVTx-300) an MPLA-like compound.

FIG. 10: Concentration of IL-18 in Rat Serum at Day 7 Post UUO Procedure in a rat model of acute kidney injury and chronic kidney disease in response to daily treatment with PHAD (REVTx-300) an MPLA-like compound.

### DETAILED DESCRIPTION

### Definitions

“About” and “approximately” shall generally mean within an acceptable degree of error for the quantity measured given the nature or precision of the measurements. Typically, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value.

“Colloid” as used herein, refers to any liquid or solid composition comprising multimolecular aggregate microstructures having diameters or lengths on the scale of 1 nm to 10 um. Such microstructures include but are not limited to micelles, liposomes, vesicles, nanoparticles, microparticles, etc. The microstructures may be spherical, oval, oblong, flat, or any other shape.

“Micelles,” as used herein, is an art-recognized term and refers to particles of colloidal dimensions that exist in equilibrium with the molecules or ions in solution from which it is formed. It is an aggregate (or supramolecular assembly) of molecules dispersed in a liquid, forming a colloidal suspension (also known as associated colloidal system). A typical micelle in water forms an aggregate with the hydrophilic “head” regions in contact with surrounding solvent, sequestering the hydrophobic single-tail regions in the micelle center.

“Liposome,” as used herein, is an art-recognized term and refers to a spherical vesicle having at least one lipid bilayer. Liposomes can be prepared by disrupting biological membranes (such as by sonication).

“Vesicle,” as used herein is an art-recognized term and refers to a membranous fluid filled sac surround by a lipid bilayer.

“Nanoparticle,” as used herein, is an art-recognized term and is typically defined as a particle of matter that is between 1 and 100 nanometers (nm) in diameter. The term is sometimes used for larger particles, up to 500 nm.

“Microparticle,” as used herein, is an art-recognized term and is defined to be particles between 1 and 1000 μm in size.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof). Treating a respiratory viral infection may include: alleviation or elimination of symptoms such as runny nose, sneezing, itchy watery eyes, cough, fatigue, headache, sore throat, or congestion.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “patient,” “subject,” or “individual” are used interchangeably and refer to either a human or a non-human animal. These terms include mammals, such as humans, non-human primates, livestock animals (including bovines, porcines, etc.), companion animals (e.g., canines, felines, etc.) and rodents (e.g., mice and rats). In some embodiments, the subject is a human.

The phrase “pharmaceutically acceptable excipient” as used herein means a pharmaceutically acceptable material, composition or vehicle, such as a liquid or solid filler, diluent, lubricant, binder, carrier, humectant, disintegrant, solvent or encapsulating material, that one skilled in the art would consider suitable for rendering a pharmaceutical formulation suitable for administration to a subject. Each excipient must be “acceptable” in the sense of being compatible with the other ingredients of the formulation, as well as “pharmaceutically acceptable” as defined above. Examples of materials which can serve as pharmaceutically acceptable excipients include but are not limited to: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; silica, waxes; oils, such as corn oil and sesame oil; glycols, such as propylene glycol and glycerin; polyols, such as sorbitol, mannitol and polyethylene glycol; esters, such as ethyl oleate and ethyl laurate; agar; buffering agents; alginic acid; pyrogen-free water; isotonic saline; Ringer's solution; and other non-toxic compatible substances routinely employed in pharmaceutical formulations.

A “therapeutically effective amount” or a “therapeutically effective dose” of a drug or agent is an amount of a drug or an agent that, when administered to a subject, will have the intended therapeutic effect. The full therapeutic effect does not necessarily occur by administration of one dose and may occur only after administration of a series of doses. Thus, a therapeutically effective amount may be administered in one or more administrations. The precise effective amount needed for a subject will depend upon, for example, the subject's size, health and age, and the nature and extent of the condition being treated.

Activation of the MyD88 signaling pathway via TLR4 stimulation results in the production of pro-inflammatory cytokines including IL-1β, IL-6, TNF-α, and IL-18 (Edilova). Long term exposure to these cytokines has been associated with the inflammation processes leading to tissue damage of chronic disease. MPLA like compounds signal primarily through the TRIF pathway via TLR4 stimulation, reducing the production of pro-inflammatory cytokines, and producing anti-inflammatory cytokines such as IFN-β, IL-4, IL-10, IP-10, and TGF-β to correct this dysregulation, slowing down or arresting the progression of chronic organ disease. TLR4 mediated TRIF bias supports potential correction of cellular activities associated with certain conditions linked to cytokine dysregulation, including inflammatory diseases, possibly macrophage activation syndrome which contributes to the cytokine storm observed in sepsis and ARDS, as well as the recent observation of “inflamm-aging” which has been used to describe the observed increase in inflammation with increased age (Rea, 2018).

The inflammation process is driven by the cellular activity that is initiated in response to pro-inflammatory cytokines. A series of cellular communications are set into motion in a complex cascade of inflammatory processes in response to tissue or cellular insult. At the site of initial damage, sentinel dendritic cells (DC) become activated, which results in the generation of multiple cytokines, including IL-12 and IL-18, which in turn activate natural killer cells (NK cells). These activated NK cells are then capable of releasing copious amounts of highly cytotoxic IFN-γ, which ends in cell death (Zwirner). Chronic DC and NK cell activation induces pro-inflammatory cytokine production which leads to a cytotoxic environment, contributing to an inflammatory state.

While not being bound by theory, MPLA is not believed to completely halt the production of pro-inflammatory cytokines; rather MPLA augments host resistance during inflammatory events by attenuation of the production of proinflammatory cytokines, which enables MPLA to fight infection while ameliorating tissue and organ damage (Watts). The decreased inflammatory signaling from TLR4 bound MPLA coupled with minimal impairment of the immunostimulatory adjuvant effect on T cells (Thompson et al., 2005; Mata-Haro et al., 2007) suggest that MPLA may prove safe and effective to use as a single-agent therapy for certain chronic inflammatory conditions.

A representative example of an MPLA-like compound, also a representative example of major species in bacterially derived MPLA, structure of synthetic phosphorylated, hexaacyl disaccharide (PHAD), is shown below:

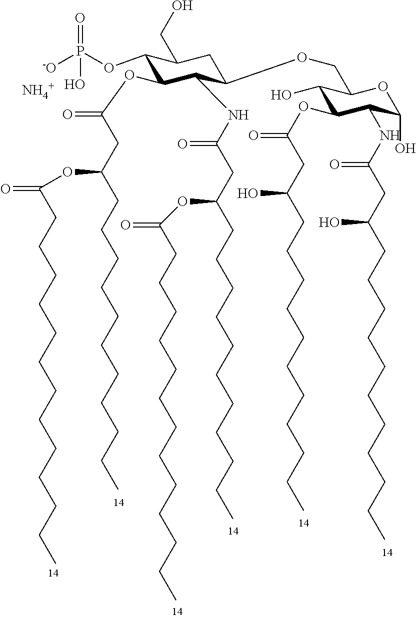

The common feature of all MPLA-like compounds is the monophosphorylated disaccharide. The degree of acylation can vary ranging from as few as 4 acyl groups to as many as 9 acyl groups. In addition, the length of each acyl chain (e.g. the number of carbons) can vary from about 8 to about 20 carbons.

MPLA-like compounds may be either synthetic or biologically derived as described (e.g. from hydrolysis of bacterial cell walls). In certain preferred embodiments, the MPLA is selected from phosphorylated hexaacyl disaccharide (PHAD), PHAD-504, 3D-(6-acyl)-PHAD, 3D-PHAD, and any combination thereof. In certain preferred embodiments, the MPLA is PHAD.

In some embodiments of the invention, the loss of organ function associated with a chronic organ disease can be treated by administering to a subject an effective amount of a TLR4 agonist. In certain embodiments, the TLR4 agonist is an MPLA-like compound. In certain preferred embodiments, the MPLA-like compound is synthetic and is selected from phosphorylated hexaacyl disaccharide (PHAD), 3-deacyl phosphorylated hexaacyl disaccharide (3D-PHAD), 3D-(6-acyl) phosphorylated hexaacyl disaccharide (3D(6-acyl) PHAD) or pharmaceutically acceptable salts thereof. In other embodiments, one or more synthetic MPLAs are co-administered together.

In some preferred embodiments, the TLR4 agonist selectively stimulates the TRIF pathway over the Myeloid differentiation primary response 88 (MyD88) pathway.

### Chronic Diseases and the Innate Immune System

Chronic organ disease is characterized by the progressive loss of organ function. Many chronic diseases of vital organs exist including chronic kidney disease (CKD), non-alcoholic steatohepatitis (NASH), osteoarthritis (OA), rheumatoid arthritis (RA), irritable bowel syndrome (IBS), pulmonary fibrotic diseases, heart diseases, etc. Many of these chronic diseases are a result of a single stress or repeated stress by either internal or external stimulus.

Chronic disease of an organ, due to chronic inflammation and subsequent fibrosis, follows a pattern of perpetual and ongoing destruction of living functional cells and subsequent replacement by the non-functional protein, collagen, resulting in fibrosis (scar tissue). The establishment of fibrosis and subsequent death of the organ is driven by ongoing inflammatory processes associated with the innate immune response. Redirection of the innate immune response from a pro-inflammatory state to an anti-inflammatory (non-inflammatory, or protective) state may slow down or halt the progressive destruction and scarring of organ tissue, allowing the healing process to take place.

The current invention contemplates using MPLA-like compounds as treatment to redirect the innate immune response from a pro-inflammatory state to an anti-inflammatory state, to restore a more normal level of function. Activation of the MyD88 signaling pathway via TLR4 stimulation results in the production of pro-inflammatory cytokines including IL-1β, IL-6, TNF-α, and IL-18 (Edilova). Long term exposure to these cytokines has been associated with the inflammation processes leading to tissue damage of chronic disease. MPLA-like compounds signal primarily through the TRIF pathway via TLR4 stimulation, reducing the production of pro-inflammatory cytokines, and producing anti-inflammatory cytokines such as IFN-β, IL-4, IL-10, IP-10, and TGF-β to correct this dysregulation, slowing down or arresting the progression of chronic organ disease.

TLR4 mediated TRIF bias supports potential correction of cellular activities associated with certain conditions linked to cytokine dysregulation, including inflammatory diseases, possibly macrophage activation syndrome, which contributes to the cytokine storm observed in sepsis and ARDS, as well as the recent observation of “inflamm-aging” which has been used to describe the observed increase in inflammation with increased age (Rea, 2018).

The inflammation process is driven by the cellular activity that is initiated in response to pro-inflammatory cytokines. A series of cellular communications are set into motion in a complex cascade of inflammatory processes in response to tissue or cellular insult. At the site of initial damage, sentinel dendritic cells (DC) become activated, which results in the generation of multiple cytokines, including IL-12 and IL-18, which in turn activate natural killer cells (NK cells). These activated NK cells are then capable of releasing copious amounts of highly cytotoxic IFN-g, which ends in cell death (Zwirner). Chronic DC and NK cell activation induces pro-inflammatory cytokine production which leads to a cytotoxic environment, contributing to an inflammatory state.

Efficient production of pro-inflammatory cytokines is dependent on the assembly of the inflammasome. Inflammasomes are multi-protein structures formed in the cytoplasm of activated innate immune cells that lead to the maturation of IL-1β and IL-18 from inactive pro-proteins to their active, mature forms (Chilton). Once the inflammasome has been established, production of pro-inflammatory cytokines is enabled via the MyD88 pathway.

The relatively low level of MyD88-driven activity observed as a result of MPLA mediated TLR4 activation is likely due to failure of inflammasome assembly (Embry et al., 2011). Relative to LPS-mediated responses, MPLA generates reduced levels of IL-1β, IL-6, and TNF-α, (Guo, Chentouh, Watts). These reduced levels of pro-inflammatory cytokines may create a deficiency in the signals required to activate DC, which prevents the generation of IL-12 and IL-18, the signals required to activate NK cells, closing off the feedback loop of the IFN-γ mediated tissue damage associated with inflammatory disease.

In some embodiments, the TLR4 agonists described herein selectively activate the TRIF pathway over the MyD88 pathway. In certain embodiments, the TLR4 agonist has an EC₅₀ for activating the TRIF pathway and its EC₅₀ for activating the MyD88 pathway have a ratio of about 1.1:1 to greater than 100,000:1. In some embodiments, the ratio of these EC₅₀s is is greater than about 1.1:1, greater than about 1.5:1, greater than about 2:1, greater than about 5:1, greater than about 10:1, greater than about 100:1, greater than about 200:1, greater than about 500:1, greater than about 1000:1, greater than about 5,000:1, greater than about 10,000:1, or greater than about 100,000:1. In some embodiments, the ratio of these EC₅₀s lies in a range of about 1:1 to about 100,000:1, about 1.1:1 to about 50,000:1, about 1.1:1 to about 10,000:1, about 1.1:1 to about 1,000:1, about 1.1:1 to about 100:1, about 1.1:1 to about 10:1, or about 1.1:1 to about 5:1.

Inflammation is a needed process in response to injury. Inflammation allows for infiltration of macrophages, T cells, and B cells to help fight injury or infection, cellular excavation of pathogen and cellular debris as a result of tissue damage, and ultimately resolution of the injury or infection. MPLA does not completely halt the production of pro-inflammatory cytokines; rather MPLA augments host resistance during inflammatory events by attenuation of the production of proinflammatory cytokines, which enables MPLA to fight infection while ameliorating tissue and organ damage (Watts). For example, the low amounts of IL-12 produced by MPLA treated DC are highly significant when compared with those produced by untreated DC. These low amounts of IL-12, although incapable of fully activating NK cells, is sufficient to induce an efficient activation of T cells (Ismaili). The decreased inflammatory signaling from TLR4 bound MPLA coupled with minimal impairment of the immunostimulatory adjuvant effect on the initial clonal expansion of T cell (Thompson et al., 2005; Mata-Haro et al., 2007) suggest that MPLA may prove safe and effective to use as a single-agent therapy for certain chronic inflammatory conditions.

In some embodiments, the loss of organ function associated with a chronic organ disease due to inflammation and fibrosis can be treated by administering to a subject an effective amount of a TLR4 agonist. In some embodiments, the TLR4 agonist is an MPLA-like compound. In some preferred embodiments the MPLA-like compound is synthetic and is selected from phosphorylated hexaacyl disaccharide (PHAD), 3-deacyl phosphorylated hexaacyl disaccharide (3D-PHAD), 3D-(6-acyl) phosphorylated hexaacyl disaccharide (3D(6-acyl) PHAD) or pharmaceutically acceptable salts thereof. In other embodiments, one or more synthetic MPLAs are co-administered together.

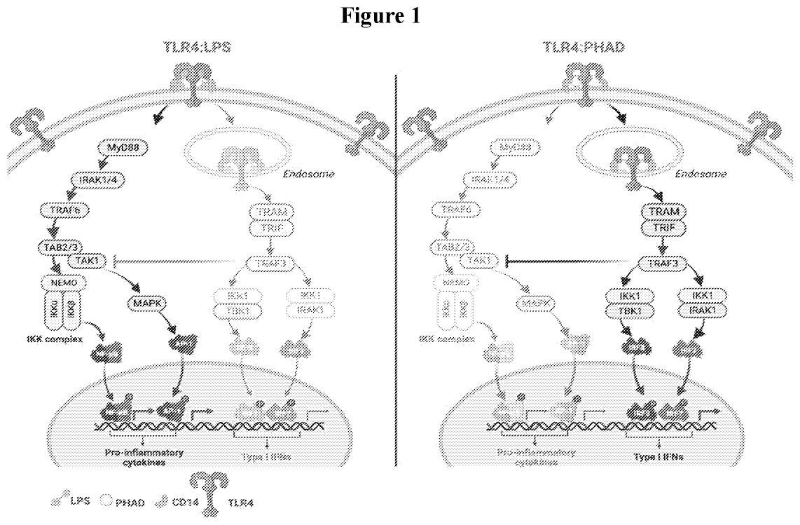

Monophosphorolipid A (MPLA)-like compounds, can selectively stimulate TLR4 to activate the TRIF pathway leading to the production of protective cytokines (FIG. 1). These protective cytokines have the ability to shift the immune response from a damaging inflammatory response to a more protective anti-inflammatory response ultimately restoring balance and normal function to the innate immune response.

In other embodiments, long term loss of organ function that is a result of acute inflammation due to internal or external stimuli can be treated by administering to a subject an effective amount of a TLR4 agonist. In some embodiments, the TLR4 agonist is an MPLA-like compound. In some preferred embodiments, the MPLA is synthetic and is selected from phosphorylated hexaacyl disaccharide (PHAD), 3-deacyl phosphorylated hexaacyl disaccharide (3D-PHAD), 3D-(6-acyl) phosphorylated hexaacyl disaccharide (3D(6-acyl) PHAD) or pharmaceutically acceptable salts thereof. In other embodiments, one or more synthetic MPLAs are co-administered together.

In some preferred embodiments, the source of acute inflammation includes one or more of the following non-limiting examples: toxicity from a medication, toxicity from chemotherapy, ischemia, trauma, cancer, and infection.

### Chronic Kidney Disease (CKD)

Kidney disease is a major public health problem, affecting ˜10% of populations in industrialized countries (1). Acute kidney injury (AKI), which affects 13.3 million people per year, may lead to chronic kidney disease (CKD). Both acute kidney injury (AKI) and chronic kidney disease (CKD) are increasing worldwide (2). Progression of chronic kidney damage often leads to end stage renal disease with the need for renal replacement therapy (dialysis or transplantation), resulting in significant morbidity and mortality for affected patients.

CKD can be initiated and propagated in several ways. One prevalent condition is the high blood sugar levels associated with diabetes (either Type 1 or Type 2). High blood sugar is toxic to kidney cells creating stress which imitates the inflammatory process leading to the demise of these cells with subsequent fibrosis ultimately resulting in continuous loss of kidney function over time. High arterial blood pressure is another source of stress that initiates the inflammatory process leading to CKD.

Other causes for CKD include: Glomerulonephritis (inflammation in the glomerulus), polycystic kidney disease, autoimmune diseases (such as systemic lupus erythematosus), vesicoureteral reflux (a condition where urine flows back up to the kidneys, pyelonephritis, interstitial nephritis (inflammation of the tubules), kidney stones, obstruction in kidney or cancer can lead to kidney failure over a period of time, overuse of certain medications, drug (heroin or cocaine) abuse, chemotherapy (such as cisplatin).

Loss of kidney function can be measured by several methods known in the art including as non-limiting examples: measurement of estimated glomerular filtration rate or measurement of true glomerular filtration rate, measurement of blood creatinine, measurement of blood urea nitrogen, measurement of urine albumin, determination of the urine-albumin-to-creatinine ratio.

In some preferred embodiments, of the invention, loss of kidney function associated with chronic kidney disease progression is prevented by administering to a subject an effective amount of a TLR4 agonist. In some preferred embodiments, the TLR4 agonist is an MPLA compound. In more preferred embodiments, the MPLA compound is synthetic and is selected from phosphorylated hexaacyl disaccharide (PHAD), 3-deacyl phosphorylated hexaacyl disaccharide (3D-PHAD), 3D-(6-acyl) phosphorylated hexaacyl disaccharide (3D(6-acyl) PHAD) or pharmaceutically acceptable salts thereof. In other embodiments, one or more synthetic MPLAs are co-administered together.

In other embodiments, loss of organ function that are a result of acute inflammation due to an acute internal or external stress can be prevented by administering to a subject an effective amount of a TLR4 agonist. In some preferred embodiments, the TLR4 agonist is an MPLA compound. In other preferred embodiments, the MPLA is synthetic and is selected from phosphorylated hexaacyl disaccharide (PHAD), 3-deacyl phosphorylated hexaacyl disaccharide (3D-PHAD), 3D-(6-acyl) phosphorylated hexaacyl disaccharide (3D(6-acyl) PHAD) or pharmaceutically acceptable salts thereof. In other embodiments, one or more synthetic MPLAs are co-administered together.

In some preferred embodiments, the acute stress is an ischemic event such as during kidney surgery or during a hypotensive state such as during severe infection (sepsis). Ischemic acute kidney injury (AKI) results in ATP depletion which leads to change in epithelial and endothelial cells. This cellular change leads to a disruption, decreasing glomerular filtration rate. The cell death incurred as a result of AKI is mediated by apoptosis and necrosis. Complex cellular interactions direct activity between injury or repair, with certain inflammatory mediators contributing to persistent injury during active tubular necrosis. (Sharfuddin). In other preferred embodiments, the acute stress is due to toxicity as a result of drug overdose or substance abuse. In other preferred embodiments, the stress is due to toxicity due to chemotherapy. In preferred embodiments, the stress is induced by platinum containing chemotherapy such as cisplatin. In another preferred embodiment, the acute stress leading to AKI is due to cardiac surgery.

In the surgical setting, ischemia may be initiated intentional, such as during a procedure that requires cardiopulmonary bypass. Ischemia may be the unintentional result of an untoward complication such as intraoperative hypotension. Regardless of etiology, the ischemic event initially leaves the affected area(s) deprived of blood, oxygen, and other nutrients and then can exacerbate to injury when the blood supply returns to the site along with reactive oxygen species (ROS) and other constituents that cause oxidative stress to the tissues.

Ischemic preconditioning and the resultant ischemic tolerance in various organs (e.g., brain, heart, liver, intestine, skeletal muscle) is an adaptive defense mechanism in which a sublethal ischemic event or exogenous stimulus (e.g., lipopolysaccharide [LPS] or monophosphoryl lipid A [MPLA]-like compounds) results in resistance to lethal ischemia. Ischemic preconditioning prevents injury upon exposure to a subsequent ischemic event by reducing excitotoxicity, apoptosis, and inflammation, thereby, protecting mitochondria and increasing anti-oxidant mechanisms (Bhuiyan 2010). An intervention that can promote ischemic tolerance in absence of oxygen deprivation techniques is a safer perioperative method for ischemic preconditioning.

### Non-Alcoholic Steatohepatitis (NASH)

Nonalcoholic fatty liver disease (NAFLD) is a condition in which excess fat builds up in the liver. This buildup of fat is not caused by heavy alcohol use. When heavy alcohol use causes fat to build up in the liver, this condition is called alcohol-associated liver disease.

Two types of NAFLD are nonalcoholic fatty liver (NAFL) and nonalcoholic steatohepatitis (NASH). People typically develop one type of NAFLD or the other, although sometimes people with one form are later diagnosed with the other form of NAFLD.

NASH is the form of NAFLD in which you have inflammation of the liver and liver damage, in addition to fat in your liver. The inflammation and liver damage of NASH can cause fibrosis, or scarring, of the liver. NASH may lead to cirrhosis, in which the liver is scarred and permanently damaged. Cirrhosis can lead to liver cancer.

In some preferred embodiments of the invention, loss of liver function associated with NASH progression is prevented by administering to a subject an effective amount of a TLR4 agonist. In some preferred embodiments, the TLR4 agonist is an MPLA compound. In more preferred embodiments, the MPLA compound is synthetic and is selected from phosphorylated hexaacyl disaccharide (PHAD), 3-deacyl phosphorylated hexaacyl disaccharide (3D-PHAD), 3D-(6-acyl) phosphorylated hexaacyl disaccharide (3D(6-acyl) PHAD) or pharmaceutically acceptable salts thereof. In other embodiments, one or more synthetic MPLAs are co-administered together.

### Osteoarthritis

Osteoarthritis is the most common form of arthritis among older people, and it is one of the most frequent causes of physical disability among older adults. The disease affects both men and women. Before age 45, osteoarthritis is more common in men than in women. After age 45, osteoarthritis is more common in women. Osteoarthritis occurs when cartilage, the tissue that cushions the ends of the bones within the joints, breaks down and wears away. Inflammatory processes drive the breakdown of the cartilage and joints. In extreme cases, all of the cartilage wears away, leaving bones exposed to rub directly against each other, requiring the insertion of artificial joints if possible.

In some preferred embodiments, of the invention, loss of joint function associated with OA progression is prevented by administering to a subject an effective amount of a TLR4 agonist that. In some preferred embodiments, the TLR4 agonist is an MPLA compound. In more preferred embodiments, the MPLA compound is synthetic and is selected from phosphorylated hexaacyl disaccharide (PHAD), 3-deacyl phosphorylated hexaacyl disaccharide (3D-PHAD), 3D-(6-acyl) phosphorylated hexaacyl disaccharide (3D(6-acyl) PHAD) or pharmaceutically acceptable salts thereof. In other embodiments, one or more synthetic MPLAs are co-administered together.

### Rheumatoid Arthritis (RA)

RA is an autoimmune and inflammatory disease that mainly attacks the joints, usually many joints at once. RA commonly affects joints in the hands, wrists, and knees. In a joint with RA, the lining of the joint becomes inflamed, causing damage to joint tissue. This tissue damage can cause long-lasting or chronic pain, unsteadiness (lack of balance), and deformity (misshapenness). RA can also affect other tissues throughout the body and cause problems in organs such as the lungs, heart, and eyes.

In some preferred embodiments of the invention, loss of joint function associated with RA progression is prevented by administering to a subject an effective amount of a TLR4 agonist. In some preferred embodiments, the TLR4 agonist is an MPLA compound. In more preferred embodiments, the MPLA compound is synthetic and is selected from phosphorylated hexaacyl disaccharide (PHAD), 3-deacyl phosphorylated hexaacyl disaccharide (3D-PHAD), 3D-(6-acyl) phosphorylated hexaacyl disaccharide (3D(6-acyl) PHAD) or pharmaceutically acceptable salts thereof. In other embodiments, one or more synthetic MPLAs are co-administered together.

### Irritable Bowel Syndrome

Irritable bowel syndrome (IBS) is a chronic disorder that affects the gastrointestinal tract, causing abdominal pain, bloating, cramping, gas, diarrhea and constipation, or both. Although common, only a small percent of those with IBS have severe symptoms, such as in Crohn's disease and ulcerative colitis, in which case the persistence of mucosal inflammation has been observed at the microscopic and molecular level. In certain cases, patients may develop IBS post infection as a result of infective gastroenteritis, which can contribute to systemic inflammation, perpetuating a cycle of chronic, low grade, subclinical inflammation. Upregulated IL-1ß has been observed in rectal biopsies in patients with postinfectious IBS. Correction, or normalization, of cytokine secretion may reduce not only the inflammation that contributes to IBS, but may also help re-establish healthy populations of the gut microflora.

In some preferred embodiments of the invention, reduction in the inflammation that may contribute to IBS or inflammatory bowel disease progression is prevented by administering to a subject an effective amount of a TLR4 agonist. In some preferred embodiments, the TLR4 agonist is an MPLA-like compound. In more preferred embodiments, the MPLA-like compound is synthetic and is selected from phosphorylated hexaacyl disaccharide (PHAD), 3-deacyl phosphorylated hexaacyl disaccharide (3D-PHAD), 3D-(6-acyl) phosphorylated hexaacyl disaccharide (3D(6-acyl) PHAD) or pharmaceutically acceptable salts thereof. In other embodiments, one or more synthetic MPLAs are co-administered together.

### Pulmonary Fibrotic Disease

Pulmonary fibrotic disease including idiopathic pulmonary fibrosis is a broad term to describe inflammation and scarring (fibrosis) of lung tissue resulting in reduced lung function. Pulmonary fibrotic disease can be caused by multiple factors including long-term exposure to toxins and pollutants, radiation and radiation treatments, and certain drugs, such as Chemotherapy drugs. Drugs designed to kill cancer cells, such as methotrexate (Trexall, Otrexup, others) and cyclophosphamide, can also damage lung tissue. Some drugs used to treat irregular heartbeats, such as amiodarone (Cordarone, Nexterone, Pacerone), may harm lung tissue. Antibiotics such as nitrofurantoin (Macrobid, Macrodantin, others) or ethambutol can cause lung damage. Certain anti-inflammatory drugs such as rituximab (Rituxan) or sulfasalazine (Azulfidine) can cause lung damage. Certain underlying medical conditions (such as Dermatomyositis, Polymyositis, Mixed connective tissue disease, Systemic lupus erythematosus, Rheumatoid arthritis, Sarcoidosis, Scleroderma, Pneumonia) may benefit from more effective anti-inflammatory treatments.

In some preferred embodiments of the invention, loss of pulmonary function associated with pulmonary fibrotic disease progression is prevented by administering to a subject an effective amount of a TLR4 agonist. In some preferred embodiments, the TLR4 agonist is an MPLA compound. In more preferred embodiments, the MPLA compound is synthetic and is selected from phosphorylated hexaacyl disaccharide (PHAD), 3-deacyl phosphorylated hexaacyl disaccharide (3D-PHAD), 3D-(6-acyl) phosphorylated hexaacyl disaccharide (3D(6-acyl) PHAD) or pharmaceutically acceptable salts thereof. In other embodiments, one or more synthetic MPLAs are co-administered together.

### Heart Disease

Cardiovascular disease (CVD), a class of diseases that impact the heart or cardiovascular system, is responsible for 31% of all deaths and remains the leading cause of mortality worldwide. Ischemic heart disease and endomyocardial fibrosis are the primary causes of end-stage heart failure.

Fibrosis is a leading cause of morbidity and mortality in cardiac disease. Fibrotic scars of the cardiac muscle most commonly occur after myocardial infarction; however, there are various other conditions promoting cardiac fibrosis such as hypertensive heart disease, diabetic hypertrophic cardiomyopathy and idiopathic dilated cardiomyopathy [4,5].

In some preferred embodiments of the invention, loss of cardiac function associated with cardiac tissue fibrosis is prevented by administering to a subject an effective amount of a TLR4 agonist. In some preferred embodiments, the TLR4 agonist is an MPLA compound. In more preferred embodiments, the MPLA compound is synthetic and is selected from phosphorylated hexaacyl disaccharide (PHAD), 3-deacyl phosphorylated hexaacyl disaccharide (3D-PHAD), 3D-(6-acyl) phosphorylated hexaacyl disaccharide (3D(6-acyl) PHAD) or pharmaceutically acceptable salts thereof. In other embodiments, one or more synthetic MPLAs are co-administered together.

### MPLA-Like Compounds

The pharmaceutical compositions of the present disclosure comprise a monophosphoryl lipid A (MPLA)-like compounds. MPLA was originally isolated from lipopolysaccharide obtained from gram-negative bacterial cell walls:

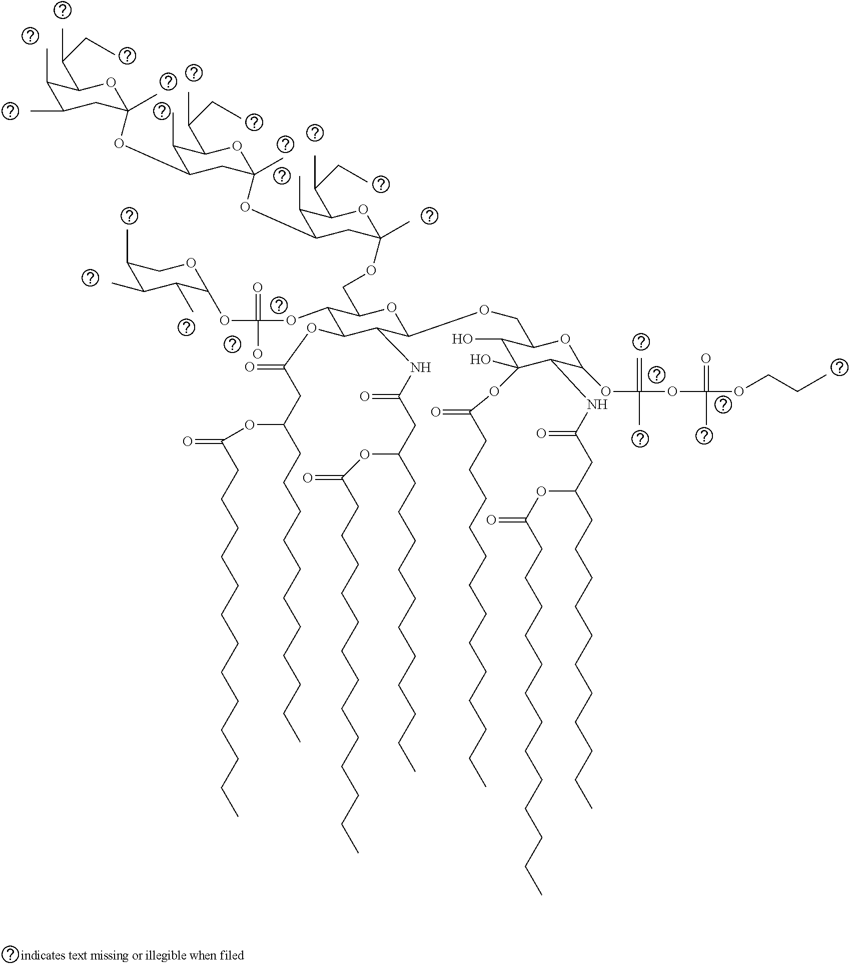

Bacterially derived MPLA is typically a mixture of several different species. shows one of the predominant species of bacterial derived MPLA. As an example, MPLA may be derived from Salmonella minnesota R595 lipopolysaccharides. As will be understood, MPLA may also be derived from other Salmonella species. The bacterial LPS may be processed via sequential acid and alkaline hydrolysis steps to remove polysaccharide side chains, phosphate groups, and to partially remove a portion of the acetyl side groups. The crude MPLA may then be purified. The final MPLA product is a mixture of heptaacyl-, hexaacyl-, and pentaacyl-monophosphorylated glucosamine disaccharide linked βlà6. Diaacetyl, triaacetyl, and tetraacetyl, if present, are considered impurities. The acylated lipids vary and include lauroyl, myristoyl, and palmitoyl. While the relative ratio of each species can vary from batch to batch, the predominant species produced are the hexaacylated disaccharide products.

The major species found in bacterially-derived MPLA have been chemically synthesized and have comparable immunostimulatory properties to the bacterially-derived material. Examples of synthetic MPLA compounds suitable for use in the present invention include phosphorylated hexaacyl disaccharide (PHAD®) (also known as glucopyranosyl lipid A, or GLA), 3D-PHAD (or 3-acyl-PHAD) (also known as monophosphoryl 3-deacyl lipid A):

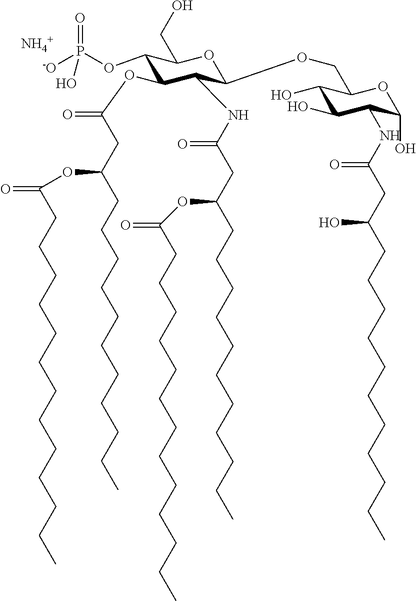

and 3D-(6-acyl) PHAD (or 3,6-acyl PHAD):

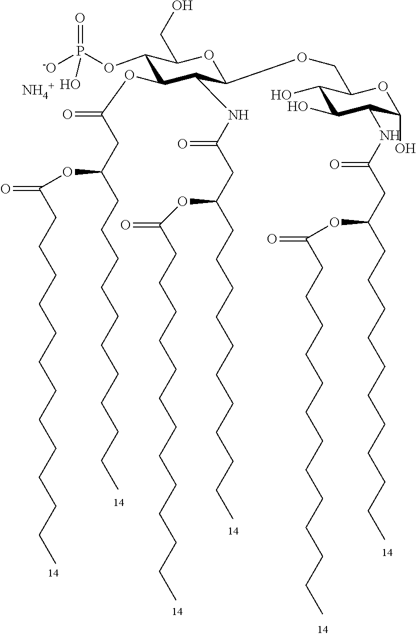

Synthetic variations of MPLA that are also suitable and within the scope of the invention include those wherein the fatty acid chain length varies between 10-20 carbons and those wherein the degree of acylation is penta-, hexa-, or hepta-.

PHAD is chemically equivalent to a major component of bacterially-derived MPLA. PHAD is also equivalent to bacterially-derived MPLA in biologic effect.

### Dosage Forms and Routes of Administration

MPLA-like compounds can be administered by several different routes. The choice of the route is dependent on multiple factors including the need (or not) for systemic exposure, the desire for the MPLA-like compound to reach a particular organ quickly, patient tolerability and compliance.

Methods of systemic delivery include those methods known in the art that provide delivery of the active molecule (e.g. the drug) to the circulatory system with distribution throughout the body. Systemic delivery methods include intramuscular, intravenous, subcutaneous, intraperitoneal, sublingual, and oral. As will be understood, any method of systemic delivery is suitable for use with the invention. Particularly suitable methods of systemic delivery include oral, intramuscular and intravenous delivery.

In some embodiments, it may desirable to only have the drug interact with the mucosal tissue and for there to no or minimally systemic exposure. Methods for mucosal delivery include those methods known in the art that provide delivery of the active molecule to mucous membranes. Mucosal delivery methods include intranasal, intrabuccal, sublingual, and oral. Particularly suitable methods for mucosal delivery include intranasal delivery.

In these embodiments, the composition comprising the MPLA compound may be formulated to be delivered to the nasal passages or nasal vestibule of the subject as droplets, an aerosol, micelles in solution, lipid or liquid nanospheres, liposomes, lipid or liquid microspheres, a solution spray, or a powder. The composition can be administered by direct application to the nasal passages or may be atomized or nebulized for inhalation through the nose or mouth.

In some embodiments, the method comprises administering a nasal spray, medicated nasal swab, medicated wipe, nasal drops, or aerosol to the subject's nasal passages or nasal vestibule. To this end, viscosity modifying agents that may be deployed to optimize the product for the application format may include cetyl alcohol, stearyl alcohol, carnauba wax, stearic acid, xanthan gum, magnesium aluminum silicate, gelatin, carbomer, poloxamers, PEGs, waxes, starches, castor oil derivatives, fatty acids, fatty alcohols, and lecithin.

In some embodiments, the compositions of the present invention can be delivered using a small needle-free nasal spray device, which can allow (self) administration with little or no prior training to deliver a desired dose. The apparatus can comprise a reservoir containing a quantity of the composition. The apparatus may comprise a pump spray for delivering one or more metered doses to the nasal cavity of a subject. The device may advantageously be single-dose use or multi-dose use. It further may be designed to administer the intended dose with multiple sprays, e.g., two sprays, e.g., one in each nostril, or as a single spray, e.g., in one nostril, or to vary the dose in accordance with the body weight or maturity of the patient. In some embodiments, nasal drops may be prepacked in pouches or ampoules that may be opened immediately prior to use and squeezed or squirted into the nasal passages. In some embodiments, the nasal spray or drops may be accomplished by time of use reconstitution of the product powder with an aqueous vehicle immediately prior to administration. In some embodiments, the nasal spray or drops may be accomplished by reconstitution of the solid drug product powder contained in a suitable delivery device using an aqueous vehicle some time period in which the drug product is deemed stable in solution format prior to patient administration.

In certain embodiments, the compositions are suitable for parenteral administration to a mammal, most preferably by injection or intravenous infusion, and in some embodiments the compositions may comprise one or more pharmaceutically acceptable excipients. Suitable excipients include pharmaceutically acceptable buffers, stabilizers, local anesthetics, and the like. The composition may be adapted for direct injection or intravenous infusion, or for addition to an intravenous drip solution for gradual infusion, through appropriate use of excipients and packaging and delivery means well known in the art.

In other embodiments, the invention provides a pharmaceutical package, comprising a vial or ampoule containing an MPLA-like compound in the form of a reconstitutable powder or a solution suitable for injection or infusion, together with instructions for administering the composition to a patient in need thereof. Instructions include but are not limited to written and/or pictorial descriptions of: the active ingredient, directions for diluting the composition to a concentration suitable for administration, suitable indications, suitable dosage regimens, contraindications, drug interactions, and any adverse side-effects noted in the course of clinical trials.

In alternative embodiments, the pharmaceutical package may comprise a plastic bag containing from 100 ml to 2 L of a pharmaceutical composition of the invention, in the form of a solution suitable for intravenous administration, together with instructions as described above.

In alternative embodiments, a pharmaceutical composition of the invention may be in a form adapted for oral dosage, such as for example a syrup or palatable solution; a form adapted for topical application, such as for example a cream or ointment; or a form adapted for administration by inhalation, such as for example a microcrystalline powder or a solution suitable for nebulization. Methods and means for formulating pharmaceutical ingredients for alternative routes of administration are well-known in the art, and it is to be expected that those skilled in the relevant arts can adapt these known methods to the MPLA-like compounds and formulations described in the present invention.

The present invention provides pharmaceutically acceptable compositions comprising a therapeutically effective amount of one or more MPLA-like compounds, formulated together with one or more pharmaceutically acceptable excipients. The pharmaceutical compositions of the present invention may be formulated for administration in solid or liquid form, including forms adapted for oral administration, for example, aqueous or non-aqueous solutions or suspensions, tablets, powders, and granules; administration by inhalation, for example, aerosols, solutions for nebulization, or dry powders; parenteral administration, for example sterile solutions or suspensions; topical application, for example lotions, creams, ointments or sprays; ophthalmic administration; or intravaginal or intrarectal administration, for example pessaries, suppositories, creams or foams. Preferably, the pharmaceutical preparation is adapted for parenteral administration, more preferably it is a non-pyrogenic solution adapted for intravenous administration.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention may optionally be scored or prepared with coatings and shells, such as enteric coatings and other coatings well known in the pharmaceutical-formulating art. They may also be formulated so as to provide slow or controlled release of the modified therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally in a delayed manner. Examples of embedding compositions that can be used include polymeric substances and waxes. The MPLA-like compound can also be in micro-encapsulated form, if appropriate, with one or more of the above-described excipients.

Liquid dosage forms for oral administration of MPLA-like compounds include pharmaceutically acceptable emulsions, microemulsions, solutions, suspensions, syrups and elixirs. In addition to the MPLA-like compound, the liquid dosage forms may contain inert diluents commonly used in the art, such as, for example, water or other solvents, solubilizing agents and emulsifiers.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In dry powder formulations adapted for inhalation, the particle size of the particulate medicament should be such as to permit inhalation of substantially all of the medicament into the lungs upon administration of the aerosol formulation and will thus desirably be less than 20 microns, preferably in the range 1 to 10 microns, more preferably 1 to 5 microns. The particle size of the medicament may be reduced by conventional means, for example by milling or micronisation. The aerosol formulation preferably contains 0.5-30% w/w of an MPLA-like compound relative to the total weight of the formulation.

The propellant may optionally contain an adjuvant having a higher polarity and/or a higher boiling point than the propellant. Polar adjuvants which may be used include (e.g. C₂₋₆) aliphatic alcohols and polyols such as ethanol, isopropanol and propylene glycol, preferably ethanol. In general, only small quantities of polar adjuvants (e.g. 0.05-3.0% w/w) may be required to improve the stability of the dispersion. However, the formulations of the invention are preferably substantially free of polar adjuvants, especially ethanol. Suitable propellants include trichlorofluoromethane (propellant 11), dichlorodifluoromethane (propellant 12), dichlorotetrafluoroethane (propellant 114), tetrafluoroethane (propellant 134a) and 1,1-difluoroethane (propellant 152a), saturated hydrocarbons such as propane, n-butane, isobutane, pentane and isopentane, and alkyl ethers such as dimethyl ether. In general, up to 50% w/w of the propellant may comprise a volatile adjuvant, for example 1 to 30% w/w of a volatile saturated C₁-C₆ hydrocarbon.

The aerosol formulations according to the invention may optionally comprise one or more surfactants that are physiologically acceptable upon administration by inhalation.

For administration by inhalation, the drug is suitably inhaled from a nebulizer, from a pressurized metered dose inhaler or as a dry powder from a dry powder inhaler optionally using gelatin, plastic or other capsules, cartridges, blister packs and/or strips.

Administration of medicament may be indicated for the treatment of mild, moderate or severe acute or chronic symptoms or for prophylactic treatment. It will be appreciated that the precise dose administered will depend on the age and condition of the patient, the particular particulate medicament used and the frequency of administration and will ultimately be at the discretion of the attendant physician. Typically, administration will range from one or to four or more times daily.

For use in dry powder inhalers, the active ingredient can be modified by spray drying or compression to form a powder with suitable flow properties. More commonly a diluent or carrier is added which is generally non-toxic and inert to the medicament. Examples of such carriers are polysaccharides e.g. starch and cellulose, dextran, lactose, glucose, mannitol, and trehalose. The carrier can be further modified by the addition of surface modifiers, pretreatment to form low rugosity particles, addition of glidants, and flavor masking or modifying agents.

Pharmaceutical compositions of this invention suitable for parenteral administration comprise an MPLA-like compound in combination with one or more pharmaceutically acceptable sterile isotonic aqueous or non-aqueous solutions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions.

Examples of pharmaceutically acceptable antioxidants include but are not limited to ascorbic acid, cysteine hydrochloride, sodium metabisulfite, sodium sulfite, ascorbyl palmitate, butylated hydroxyanisole (BHA), butylated hydroxytoluene (BHT), propyl gallate, alpha-tocopherol, and chelating agents such as citric acid, ethylenediamine tetraacetic acid (EDTA), sorbitol, tartaric acid, phosphoric acid, and the like.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

Dosage forms for the topical or transdermal administration of a compound of this invention include powders, sprays, ointments, pastes, creams, lotions, gels, solutions, patches and inhalants. The MPLA-like compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Ophthalmic formulations, eye ointments, powders, solutions and the like, are also contemplated as being within the scope of this invention.

Formulations of the present invention which are suitable for vaginal administration include pessaries, tampons, creams, gels, pastes, foams or spray formulations, containing such carriers as are known in the art to be appropriate. Such formulations may be prepared, for example, by mixing one or more MPLA-like compounds with one or more suitable nonirritating excipients comprising, for example, cocoa butter, polyethylene glycol, or a suppository wax, which is solid at room temperature but liquid at body temperature and, therefore, will melt in the rectum or vaginal cavity and release the MPLA-like compound.

### Therapeutic Doses and Regimens

In certain embodiments, an MPLA-like compound is administered in a total dose between 0.001 to 100 milligrams depending on the route of administration (e.g. parenteral, oral) and treatment target (acute vs chronic disease). In preferred embodiments, the total dose administered is between 50 and 1000 micrograms. In particularly preferred embodiments, the total dose is about 100 to 500 micrograms. In other preferred embodiments the total dose is about 5-20 mg. In another preferred embodiment the total dose is about 50-100 mg.

In other embodiments of the invention, an MPLA-like compound is given as a single dose. In other embodiments, MPLA-like compound is given multiple times. In the case of multiple doses, MPLA-like compounds may be given daily, bi-weekly, weekly or monthly. The exact frequency of dosing and the dose required at each interval will depend on multiple factors including the type of chronic organ disease being treated, the rate of disease progression and patient tolerability. Early in the treatment phase, the dose and/or dosing frequency may be greater with a gradual decrease in both dose and/or dosing frequency as the progression of disease diminishes to all for maintenance of a steady state (lack of disease progression)

### Compositions and Methods of Preparation

In certain embodiments, the pharmaceutical composition is an aqueous composition. In some such embodiments, the pharmaceutical compositions may contain an organic solvent. In certain embodiments, the organic solvent may comprise up to 15% of the total end volume of the administered drug product solution. In preferred embodiments, the organic solvent may comprise up to 5% of the total end volume of the administered drug product solution.

In certain embodiments, the organic solvent is miscible with water, such as an organic solvent selected from an alcohol, glycerin, low molecular weight polyethylene glycol, and low molecular weight poloxamers. In certain preferred embodiments, the organic solvent is an alcohol, e.g., methanol, ethanol, isopropanol, t-butanol, or preferably ethanol.

In certain embodiments, the pharmaceutical compositions further comprise one or more surfactants. In certain embodiments, the one or more surfactants are selected from carboxymethyl cellulose, dodecyltrimethylammonium bromide (DTAB), n-dodecyl octa (ethylene oxide) (C12E8), n-dodecyl tetra (ethylene oxide) (C12E4), dioctanoyl phosphatidylcholine (C8-lecithin), Polyoxyl 35 castor oil, Cremophor EL (CrEL), Octaethylene glycol monododecyl ether (C12E8), hexadecyltrimethylammonium bromide (CTAB), polypropylene oxide (PPO), polyethylene oxide (PEO), PEO-poly(D,L-lactic acid-co-caprolactone) (PEO-PDLLA), and sodium dodecyl sulfate (SDS). In certain preferred embodiments, the one or more surfactants is carboxymethyl cellulose.

In certain embodiments, the pharmaceutical compositions may contain excipients, additives, bulking agents, and mucoadhesive agents. These may include mannitol, trehalose, cyclodextrin, and hydroxypropyl methylcellulose (HPMC). In preferred embodiments, the excipients HP-β-Cyclodextrin and trehalose are employed.

In certain embodiments, the pharmaceutical compositions further comprise a phospholipid selected from phosphatidic acid (PA), phosphatidylcholine (PC), phosphatidylglycerol (PG), phophatidylethanolamine (PE), phophatidylinositol (PI), phosphatidylserine (PS), sphingomyelin (including brain sphingomyelin), lecithin, lysolecithin, lysophosphatidylethanolamine, cerebrosides, diarachidoylphosphatidylcholine (DAPC), didecanoyl-L-alpha-phosphatidylcholine (DDPC), dielaidoylphosphatidylcholine (DEPC), dilauroylphosphatidylcholine (DLPC), dilinoleoylphosphatidylcholine, dimyristoylphosphatidylcholine (DMPC), dioleoylphosphatidylcholine (DOPC), dipalmitoylphosphatidylcholine (DPPC), distearoylphosphatidylcholine (DSPC), 1-palmitoyl-2-oleoyl-phosphatidylcholine (POPC), diarachidoylphosphatidylglycerol (DAPG), didecanoyl-L-alpha-phosphatidylglycerol (DDPG), dielaidoylphosphatidylglycerol (DEPG), dilauroylphosphatidylglycerol (DLPG), dilinoleoylphosphatidylglycerol, dimyristoylphosphatidylglycerol (DMPG), dioleoylphosphatidylglycerol (DOPG), dipalmitoylphosphatidylglycerol (DPPG), distearoylphosphatidylglycerol (DSPG), 1-palmitoyl-2-oleoyl-phosphatidylglycerol (POPG), diarachidoylphosphatidylethanolamine (DAPE), didecanoyl-L-alpha-phosphatidylethanolamine (DDPE), dielaidoylphosphatidylethanolamine (DEPE), dilauroylphosphatidylethanolamine (DLPE), dilinoleoylphosphatidylethanolamine, dimyristoylphosphatidylethanolamine (DMPE), dioleoylphosphatidylethanolamine (DOPE), dipalmitoylphosphatidylethanolamine (DPPE), distearoylphosphatidylethanolamine (DSPE), 1-palmitoyl-2-oleoyl-phosphatidylethanolamine (POPE), diarachidoylphosphatidylinositol (DAPI), didecanoyl-L-alpha-phosphatidylinositol (DDPI), dielaidoylphosphatidylinositol (DEPI), dilauroylphosphatidylinositol (“DLPI), dilinoleoylphosphatidylinositol, dimyristoylphosphatidylinositol (DMPI), dioleoylphosphatidylinositol (DOPI), dipalmitoylphosphatidylinositol (DPPI), distearoylphosphatidylinositol (DSPI), 1-palmitoyl-2-oleoyl-phosphatidylinositol (POPI), diarachidoylphosphatidylserine (DAPS), di decanoyl-L-alpha-phosphatidylserine (DDPS), dielaidoylphosphatidylserine (DEPS), dilauroylphosphatidylserine (DLPS), dilinoleoylphosphatidylserine, dimyristoylphosphatidylserine (DMPS), dioleoylphosphatidylserine (DOPS), dipalmitoylphosphatidylserine (DPPS), distearoylphosphatidylserine (DSPS), 1-palmitoyl-2-oleoyl-phosphatidylserine (POPS), diarachidoyl sphingomyelin, didecanoyl sphingomyelin, dielaidoyl sphingomyelin, dilauroyl sphingomyelin, dilinolcoyl sphingomyelin, dimyristoyl sphingomyelin, sphingomyelin, dioleoyl sphingomyelin, dipalmitoyl sphingomyelin, distearoyl sphingomyelin, 1-palmitoyl-2-oleoyl-sphingomyelin, and any combination thereof.

In certain embodiments, the pharmaceutical composition is free or substantially free of salt (e.g., NaCl).

In certain embodiments, the pharmaceutical composition has an MPLA concentration of about 1 μg/mL to about 10,000 μg/mL. In certain embodiments, the pharmaceutical composition has an MPLA concentration of about 50 μg/mL to about 200 μg/mL. In certain embodiments, the pharmaceutical composition has an MPLA concentration of about 125 μg/mL. In certain embodiments, the pharmaceutical composition has an MPLA concentration of about 250 μg/mL.

In certain embodiments, the pharmaceutical compositions further comprise a stabilizer or multiple stabilizers. In certain preferred embodiments, the stabilizer is trehalose. In other preferred embodiments, the stabilizer is HP-β-Cyclodextrin. In some most preferred embodiments, both HP-β-Cyclodextrin and trehalose are used as stabilizers.

In certain embodiments, the pharmaceutical composition is a dry powder.

In certain embodiments, the colloidal or micellar solution comprises particles having an average diameter of about 1 nm to about 1000 nm. In certain preferred embodiments, the particles have an average diameter of about 50 nm to about 500 nm. In other more preferred embodiments, the particles have an average diameter of about 100 nm to about 500 nm. In other embodiments, the particles have an average diameter of about 500 nm to about 1000 nm. In certain embodiments, the particles have an average diameter of about 10 nm to about 200 nm. In preferred embodiments, the particles have an average diameter of about 10 nm to about 100 nm. In certain embodiments, the particles have an average diameter of less than 50 nm. In certain embodiments, the particles have an average diameter of less than 100 nm. In certain embodiments, the particles have an average diameter of less than 150 nm. In certain embodiments, the particles have an average diameter of less than 200 nm. In certain embodiments, the particles have an average diameter of less than 250 nm. In certain embodiments, the particles have an average diameter of less than 500 nm.

In certain embodiments, the pharmaceutical compositions further comprise a mucoadhesive agent. In certain embodiments, the mucoadhesive agent is selected from cellulose derivatives, polyacrylates, a starch, chitosan, glycosylaminoglycans, hyaluronic acid, cellulose derivatives, polyacrylates, and any combination thereof.

In certain preferred embodiments, the pharmaceutical compositions further comprise a pH modifier, an emulsifier, a pH buffer, a tonicity modifier, a stabilizer, a preservative, a surfactant, a bulking agent, a flavorant, or any combination thereof.

In certain embodiments, the bulking agent is selected from mannitol, trehalose, chitosan, HP-β-Cyclodextrin, hydroxypropylmethylcellulose (HPMC), dextran, starch (such as pea starch), and sucrose.

In certain embodiments, the colloid comprises micelles. In certain embodiments, the colloidal suspension comprises liposomes. In certain embodiments, the colloidal suspension comprises nanoparticles. In certain embodiments, the colloidal suspension comprises microparticles.

In certain embodiments, provided are methods of preparing a pharmaceutical composition disclosed herein, comprising:
a. dissolving one or more MPLAs in an organic solvent to form an organic solvent/MPLA solution;
b. combining the organic solvent/MPLA solution with water to form a colloidal formulation comprising the one or more MPLAs.

In certain embodiments, step (a) or (b) is performed under sonication.

In certain embodiments, the organic solvent and the water are present in a volume to volume ratio of about 1:1500 to about 1:50. In certain embodiments, the organic solvent and water are present in a volume to volume ratio of about 1:1000 to about 1:100. In certain embodiments, the organic solvent and water are present in a volume to volume ratio of about 1:800.

In certain embodiments, one or more surfactants are added in step (a) or (b). In some preferred embodiments, the surfactant is carboxymethyl cellulose. In another embodiments, one or more phospholipids is added in step (a) or (b). In certain embodiments, the mixture in step (b) is lyophilized. In other embodiments, the mixture in step (b) is spray dried. In some preferred embodiments, the mixture in step (b) is stabilized with trehalose. In certain embodiments, step (a) or step (b) is performed at an elevated temperature. In certain embodiments, the elevated temperature is about 30° C. to about 50° C. In certain preferred embodiments, the elevated temperature is about 40° C. In certain embodiments, the mixture in step (b) has an MPLA concentration of about 125 μg/mL.

In certain embodiments, the mixture in step (b) has an MPLA concentration ranging from about 1 μg/mL to about 1000 μg/mL; about 20 μg/mL to about 500 g/mL, about 100 μg/mL to about 300 μg/mL, about 250 μg/mL, or about 125 μg/mL.

In certain embodiments, the spray-dried powder has an MPLA concentration of 0.25 to 10% w/w MPLA/solids in the final powder. In certain preferred embodiments, the MPLA concentration in the final powder is 0.25 to 2% w/w MPLA/solids.

In certain embodiments, the spray dried powder is reconstituted with water prior to administration to the patient.

In some embodiments, the MPLA compound is administered in a composition comprising one or more pharmaceutically acceptable excipients. The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

For example, the composition comprising the MPLA compound can be formulated for intranasal delivery as a dry powder, as an aqueous solution, an aqueous suspension, a colloid, a water-in-oil emulsion, a micellar formulation, or as a liposomal formulation.

In some embodiments, the MPLA composition comprises micelles of the MPLA compound. While not being bound by theory, it is believed that micelles enhance the activity of the MPLA. The size of the micelles, in some embodiments, is about 50 nm to about 1000 nm. The size of micelles may be measured by various techniques, including dynamic light scattering (DLS), scanning electron microscopy (SEM), and transmission electron microscopy (TEM). Accordingly, in some embodiments, the size of the micelles is about 50 nm to about 1000 nm as measured by DLS.

In certain preferred embodiments, the composition further comprises an organic solvent, such as an alcohol, glycerin, low molecular weight polyethylene glycol, a poloxamer, or any combination thereof. In some embodiments, the organic solvent is water miscible. In some embodiments, the organic solvent is an alcohol, such as methanol, ethanol, isopropanol, or t-butanol, preferably ethanol.

In some embodiments, the composition further comprises a fatty acid salt, fatty acids, a phospholipid, or any combination thereof.

In some embodiments, the composition comprises a phospholipid or a mixture of phospholipids. Examples of phospholipids include, but are not limited to, phosphatidic acid (PA), phosphatidylcholine (PC), phosphatidylglycerol (PG), phophatidylethanolamine (PE), phophatidylinositol (PI), and phosphatidylserine (PS), sphingomyelin (including brain sphingomyelin), lecithin, lysolecithin, lysophosphatidylethanolamine, cerebrosides, diarachidoylphosphatidylcholine (DAPC), didecanoyl-L-alpha-phosphatidylcholine (DDPC), dielaidoylphosphatidylcholine (DEPC), dilauroylphosphatidylcholine (DLPC), dilinoleoylphosphatidylcholine, dimyristoylphosphatidylcholine (DMPC), dioleoylphosphatidylcholine (DOPC), dipalmitoylphosphatidylcholine (DPPC), distearoylphosphatidylcholine (DSPC), 1-palmitoyl-2-oleoyl-phosphatidylcholine (POPC), diarachidoylphosphatidylglycerol (DAPG), didecanoyl-L-alpha-phosphatidylglycerol (DDPG), dielaidoylphosphatidylglycerol (DEPG), dilauroylphosphatidylglycerol (DLPG), dilinoleoylphosphatidylglycerol, dimyristoylphosphatidylglycerol (DMPG), dioleoylphosphatidylglycerol (DOPG), dipalmitoylphosphatidylglycerol (DPPG), distearoylphosphatidylglycerol (DSPG), 1-palmitoyl-2-oleoyl-phosphatidylglycerol (POPG), diarachidoylphosphatidylethanolamine (DAPE), didecanoyl-L-alpha-phosphatidylethanolamine (DDPE), dielaidoylphosphatidylethanolamine (DEPE), dilauroylphosphatidylethanolamine (DLPE), dilinoleoylphosphatidylethanolamine, dimyristoylphosphatidylethanolamine (DMPE), dioleoylphosphatidylethanolamine (DOPE), dipalmitoylphosphatidylethanolamine (DPPE), distearoylphosphatidylethanolamine (DSPE), 1-palmitoyl-2-oleoyl-phosphatidylethanolamine (POPE), diarachidoylphosphatidylinositol (DAPI), didecanoyl-L-alpha-phosphatidylinositol (DDPI), dielaidoylphosphatidylinositol (DEPI), dilauroylphosphatidylinositol (DLPI), dilinoleoylphosphatidylinositol, dimyristoylphosphatidylinositol (DMPI), dioleoylphosphatidylinositol (DOPI), dipalmitoylphosphatidylinositol (DPPI), distearoylphosphatidylinositol (DSPI), 1-palmitoyl-2-oleoyl-phosphatidylinositol (POPI), diarachidoylphosphatidylserine (DAPS), didecanoyl-L-alpha-phosphatidylserine (DDPS), dielaidoylphosphatidylserine (DEPS), dilauroylphosphatidylserine (DLPS), dilinoleoylphosphatidylserine, dimyristoylphosphatidylserine (DMPS), dioleoylphosphatidylserine (DOPS), dipalmitoylphosphatidylserine (DPPS), distearoylphosphatidylserine (DSPS), 1-palmitoyl-2-oleoyl-phosphatidylserine (POPS), diarachidoyl sphingomyelin, didecanoyl sphingomyelin, dielaidoyl sphingomyelin, dilauroyl sphingomyelin, dilinoleoyl sphingomyelin, dimyristoyl sphingomyelin, sphingomyelin, dioleoyl sphingomyelin, dipalmitoyl sphingomyelin, distearoyl sphingomyelin, 1-palmitoyl-2-oleoyl-sphingomyelin, and any combination thereof.

In certain embodiments, the phospholipid is DPPC, DOPC, cholesterol, or a mixture thereof.

In certain embodiments, compositions comprising MPLA may contain pH modifiers, pH buffers, oils/emulsifiers (e.g., squalene), tonicity modifiers, stabilizers, preservatives, detergents, flavorants, bulking agents, or secondary immunostimulatory agents. In some embodiments, the composition is a dry powder comprising a bulking agent.

Secondary immunostimulatory agents include, e.g., gonadocorticoids, deoxycholic acid, vitamin D, and beta-glucans. Suitable buffers include sodium chloride-based or potassium chloride-based solutions such as phosphate buffered saline, potassium buffered saline, or borate buffered saline. In some embodiments, the buffer may contain salts, detergents, or carbohydrates which preserve the MPLA upon drying and aid in resolubilizing the MPLA upon encounter with a liquid. Suitable carbohydrates include trehalose, sucrose, glucose, and mannose.

In some embodiments, the composition further comprises a mucoadhesive. Suitable mucoadhesives include: glycosylaminoglycans (GAGS) including chondroitin sulfate, chitosan, hyaluronic acid, cellulose derivatives, HP-B-Cyclodextrin, polyacrylates, starch, HPMC and any combination thereof.

In some embodiments, the mucoadhesive is present in the composition in an amount ranging from about 0.1 to about 50% by weight, about 25% to about 50% by weight, or about 49% by weight.

In some embodiments, the composition further comprises a sugar. Examples of sugars that may be used in the methods provided herein include, but are not limited to, sucrose, glucose, fructose, lactose, maltose, mannose, galactose, trehalose, and combinations thereof. In certain embodiments, the sugar content is about 49% by weight.

In some embodiments, the composition is an aqueous liquid. In such embodiments, the concentration of the MPLA compound in the composition may be about 1 μg/mL to about 1000 μg/mL, about 20 μg/mL to about 500 μg/mL, about 100 μg/mL to about 300 μg/mL, or about 250 μg/mL.

In certain embodiments, the formulation may contain ionic or nonionic surfactants. Suitable surfactants include poloxamer 407, poloxamer 181, dodecyltrimethylammonium bromide (DTAB), n-dodecyl octaethylene oxide (C12E8), n-dodecyl tetraethylene oxide (C12E4) and dioctanoyl phosphatidylcholine (C8-lecithin), polyoxyl 35 castor oil, cremophor EL (CrEL), octaethylene glycol monododecyl ether (C12E8), hexadecyltrimethylammonium bromide (CTAB), polypropylene oxide (PPO), polyethylene oxide (PEO), PEO-poly(D,L-lactic acid-co-caprolactone) (PEO-PDLLA), and sodium dodecyl sulfate (SDS) and any combination thereof.

In some embodiments, the MPLA formulation has a pH between 4 and 9. In certain preferred embodiments, the pH is between 5 and 8.

In certain embodiments, the formulations may be free of or substantially free of phospholipids, surfactants, salt (e.g., NaCl), and/or buffers. Substantially free means that the substance in question makes up less than 0.5%, less than 0.25%, less than 0.1%, less than 0.05%, less than 0.01%, or less than 0.005% of the composition by weight.

In some embodiments of the invention, the composition comprises an MPLA compound at a concentration between 1 and 8000 μg/mL. In certain preferred embodiments, the MPLA is present at a concentration between 20 and 500 μg/mL. In certain more preferred embodiments, the concentration is between 100 and 300 μg/mL. In certain embodiments, the concentration is 250 μg/mL, and in still other embodiments, the concentration is 125 μg/mL.

In some embodiments of the invention, the solution is formulated such that a surfactant is included at a concentration between 1 and 40% w/w, which can enhance the absorption of the drug upon administration by preventing degradation/metabolism, enhancing barrier permeability via transient opening of tight junctions, disruption of lipid bilayer packing/complexation/carrier/ion pairing and enhancing resident time/slowing down mucociliary clearance. In certain preferred embodiments, the surfactant concentration is between 1 and 25% w/w. In certain most preferred embodiments, the surfactant concentration is 15% w/w. Surfactants of interest include, but are not limited to, dipalmitoyl phosphatidyl choline, soybean lecithin, phosphatidylcholine, sodium taurocholate, sodium deoxycholate sodium, glycodeoxycholate, palmitic acid, stearic acid, and oleic acid.

In some embodiments of the invention, the composition comprises a mucoadhesive at a concentration between 0.1 to 50% w/w, which can enhance the absorption of the drug upon administration by enhancing resident time and/or slowing down mucociliary clearance. In certain preferred embodiments, the mucoadhesive is included at a concentration between 40 to 50% w/w. In certain most preferred embodiments, the mucoadhesive is included at a concentration of 49% w/w. Mucoadhesives of interest include, but are not limited to, cellulose derivatives, HP-β-Cyclodextrin polyacrylates, starch, and chitosan.

In other preferred embodiments, the composition is a powder that comprises an MPLA-like compound and one or more bulking agents. Useful compositions comprise 2.5 to 50% by weight of the MPLA-like compound and 50 to 97.5% by weight of one or more bulking agents. Preferred compositions contain 5-20% by weight of the MPLA-like compound and 80 to 95% by weight of one or more bulking agents. A particularly preferred composition contains about 10% of the MPLA-like compound and about 90% by weight of one or more bulking agents.

### Combination Therapies

A range of anti-inflammatory treatments are available for certain inflammation-related indications, such as Humira, a monoclonal antibody therapeutic which targets and removes TNF-α from circulation. As TNF-α is a contributing factor in inflammation, reduction of TNF-α reduces symptoms associated with several inflammatory conditions such as rheumatoid arthritis, eczema, and psoriasis. As Humira targets a single, specific pro-inflammatory cytokine, is does not provide wide spectrum relief due to other sources of inflammation. Monoclonal antibody treatment may require several weeks to demonstrate reduction in symptoms.

Monoclonal antibody therapy for the treatment of inflammation may provide a benefit to treatment when administered in combination with MPLA, as this would in theory provide both short and long term reduction in symptoms as a result of inflammation. In some embodiments, the monoclonal antibody is directed to target galectin-3. In some embodiments the monoclonal antibody is directed to target inflammatory cytokines including, but not limited to, IL-6, IL-23, IL-33, and/or IL-1β.

In some embodiments of the invention, the composition is administered prior to, simultaneously, or after monoclonal antibody treatment.

### EXAMPLES

In order that the invention described herein may be more fully understood, the following examples are set forth. The examples described in this application are offered to illustrate the compounds, compositions, materials, device, and methods provided herein and are not to be construed in any way as limiting their scope.

### Example 1

### Preparation of PHAD Micelles in 5% Ethanol.

1 mg of PHAD was wetted for 1 minute in 0.4 mL of 95% ethanol then sonicated for 15 minutes at 40° C. until a clear solution is formed. The solution was removed from the sonication bath and QS to 8 mL with water, resulting in a homogeneous formulation of PHAD micelles 150 nm or smaller in size. The formulation was either used as liquid or lyophilized.

### Example 2

### Preparation of PHAD Powder.

6 mg of PHAD was wetted and dissolved in 3 mL of 95% ethanol at 40° C. and sonicated for 20 minutes at 40° C. until a clear solution of 2 mg/mL PHAD was obtained. 17 mL of water at 40° C. was then added and sonicated to fully mix the bulk solution, resulting in a homogeneous formulation of PHAD micelles around 150 nm or smaller in size. 147 mg of HP-β-Cyclodextrin and 147 mg of Trehalose dihydrate were added to the solution under mixing. This solution was then spray dried to obtain a final powder at 2% w/w PHAD: 49% w/w HP-β-Cyclodextrin: 49% w/w Trehalose.

The powder is stable for extended time at ambient temperature and is readily soluble in purified water up to concentrations of about 5-10 mg/mL. This powder can be reconstituted for parental administration for example by intravenous, subcutaneous, or intramuscular route of administration, or by intranasal administration. Alternatively, this same powder can be formulated with additional excipients to form a tablet or gel for oral delivery.

### Example 3

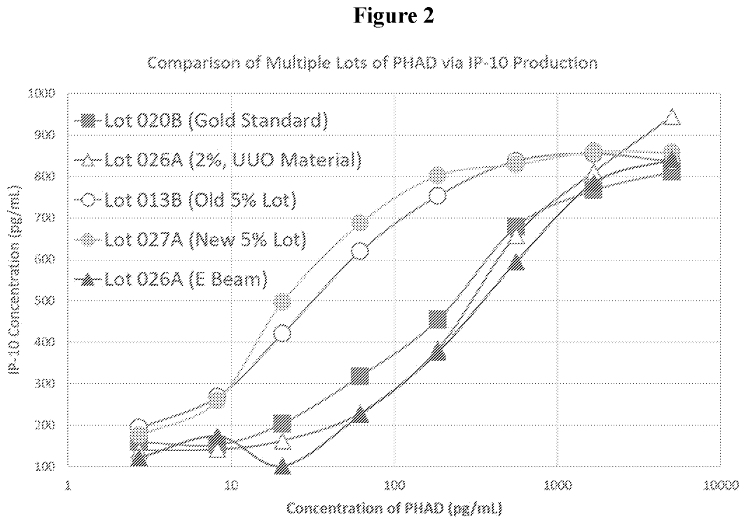

Formulations as prepared in Examples 1 and 2 show robust upregulation of IP-10, as a result of TLR4 stimulation in vitro in mouse macrophages (FIG. 2). IP-10 is an important cytokine associated with stimulation of the TRIF pathway. Evidence of IP-10 upregulation is confirmation that PHAD preparations as noted in this application are capable of selectively stimulating the TRIF pathway, which is further confirmation of the amelioration or reduction of activity mediated through the MyD88 signaling pathway, which supports the amelioration of pro-inflammatory cytokine production for MPLA as a treatment for inflammatory conditions.

### Example 4

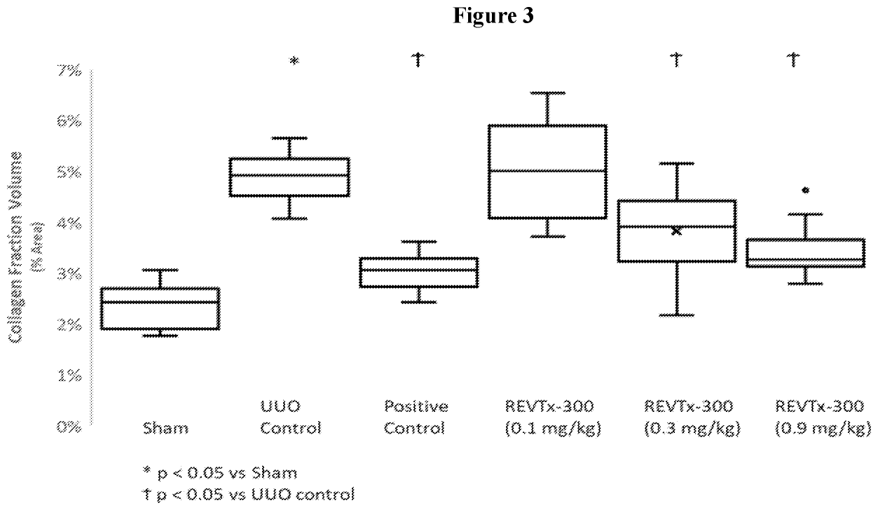

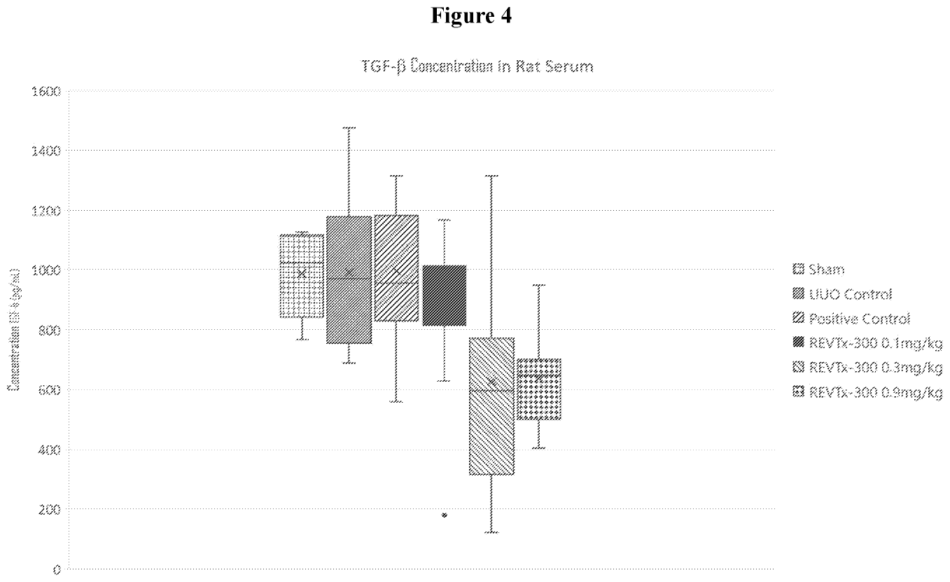

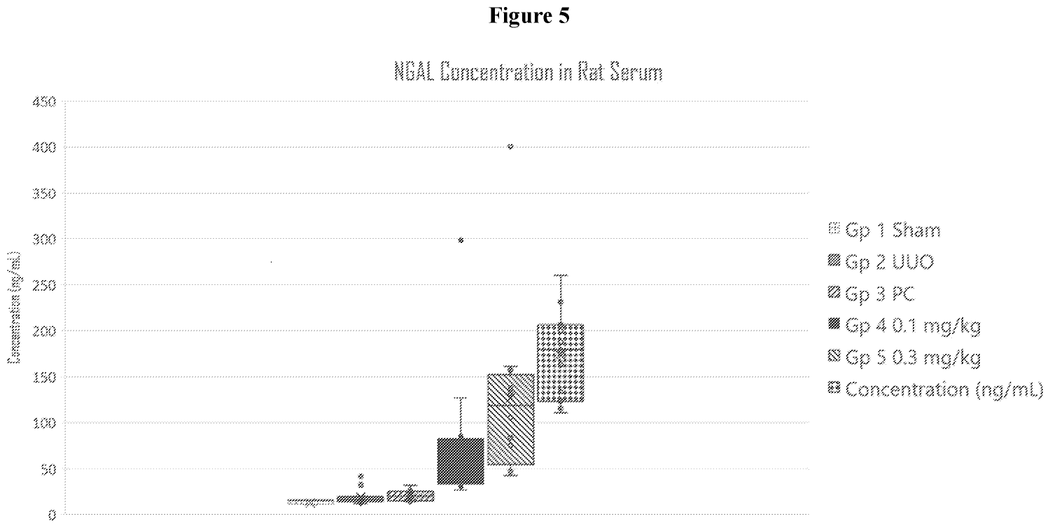

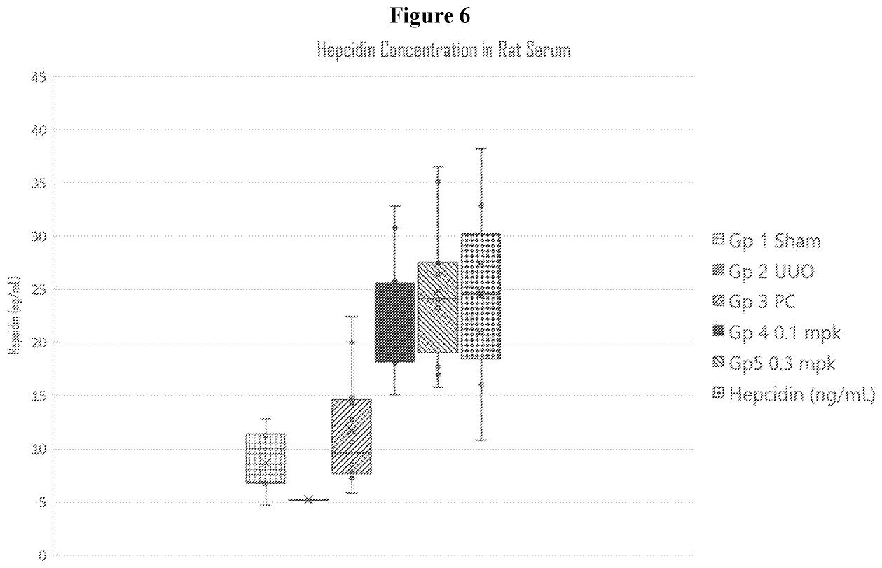

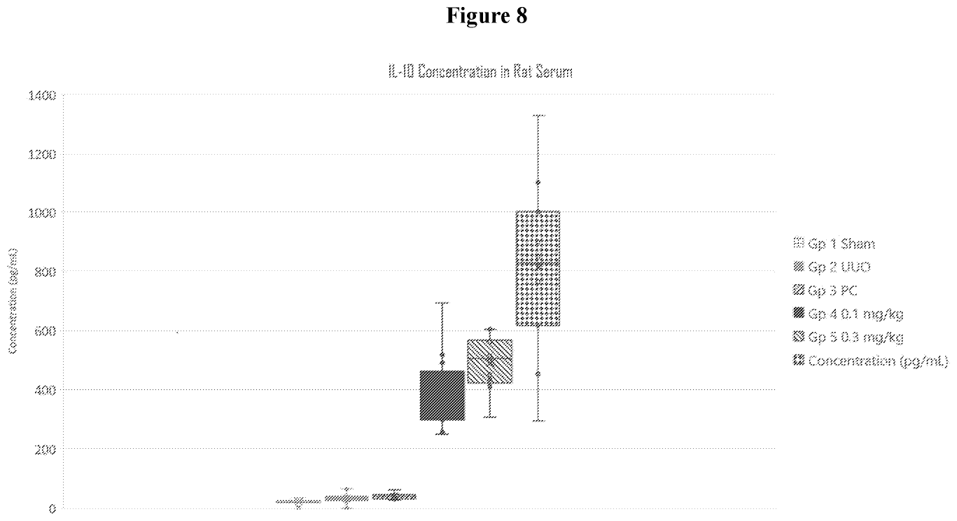

In a validated preclinical model of acute kidney injury (AKI) and chronic kidney disease (CKD), daily administration of REVTx-300, an MPLA-like compound, caused significant reduction in fibrosis and circulating transforming growth factor-β (TGF-β) in a dose dependent manner relative to the positive control group (FIGS. 3 and 4). Composite data represents the average of 3 anatomically distinct depths (10 images/depth/rat/group=˜60-65% of renal cortical area). Renal cortical fibrosis, expressed as Collagen Volume Fraction (CVF; via quantitation of PSR stained tissue sections) was increased in vehicle-treated UUO obstructed kidneys relative to sham-operated control. SB-525334 attenuated UUO-induced increases in renal cortical CVF. REVTx-300 (PHAD, and MPLA-like compound) dosed at 0.3 mg/kg and 0.9 mg/kg attenuated UUO-induced increases in renal cortical CVF. In addition, REVTx-300 significantly increased circulating anti-inflammatory interleukin-10 (IL-10) (FIG. 8), hepcidin (FIG. 6), and neutrophil gelatinase-associated lipocalin (NGAL) (FIG. 5) in all groups in a dose dependent manner, relative to the positive control group in the unilateral ureteral obstruction (UUO) model.

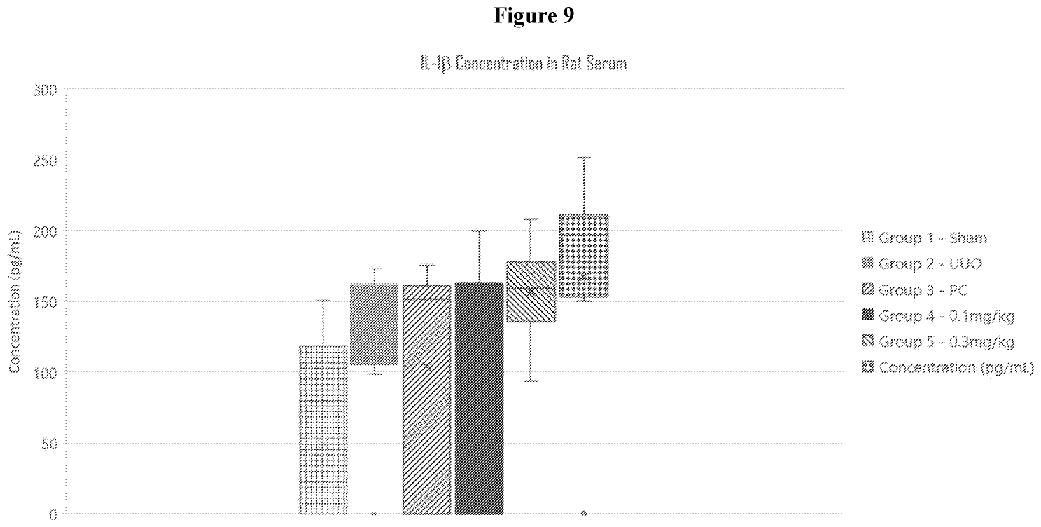

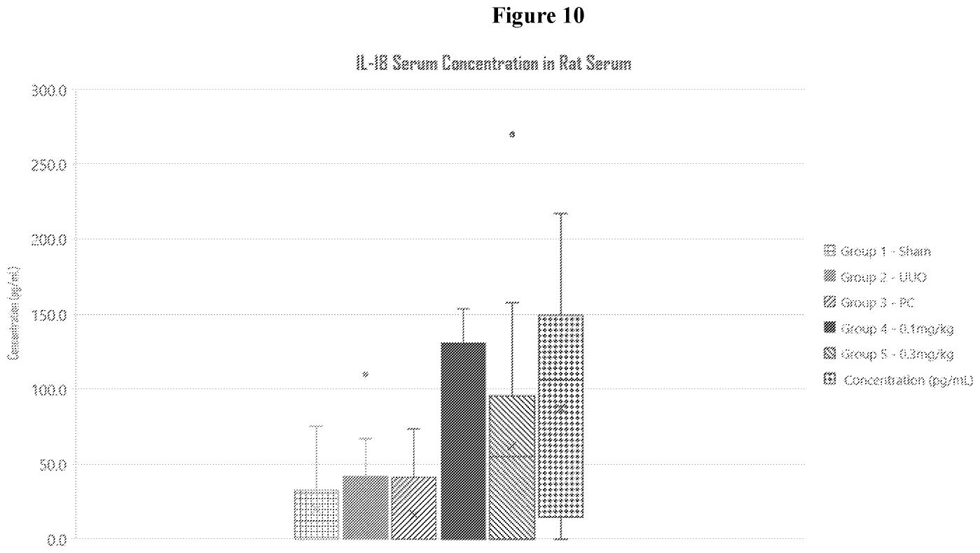

TGF-β is a key driver of fibrogenesis and contributes directly to the deposition of collagen through excessive production of extracellular matrix. IL-10 is characterized as an anti-inflammatory cytokine, due to its capability to reduce the generation of pro-inflammatory mediators. Hepcidin and NGAL sequester iron to prevent iron-mediated reactive oxygen tissue damage. There were no significant increases in markers of inflammation (no increase in IL-6, relatively minor increase in IL-1β (FIG. 9) and IL-18 (FIG. 10)). These results provide mechanistic evidence for the reduction in fibrosis observed in the UUO model in response to treatment with REVTx-300.

### REFERENCES

Bhuiyan M I, Kim Y J. Mechanisms and prospects of ischemic tolerance induced by cerebral preconditioning. Int Neurourol J. 2010; 14 (4): 203-212. doi: 10.5213/inj.2010.14.4.203
Chentouh R, Fitting C, Cavaillon J M. Specific features of human monocytes activation by monophosphoryl lipid A. Sci Rep. 2018; 8 (7096).
Chilton P M, Embry C A, Mitchell T C. Effects of differences in lipid A structure on TLR4 pro-inflammatory signaling and inflammasome activation. Front Immunol. 2012; 3 (154).
da Silva L, Neves B M, Moura L, Cruz M T, Carvalho E. Neurotensin downregulates the pro-inflammatory properties of skin dendritic cells and increases epidermal growth factor expression. Biochem Biophys Acta. 2011; 1813 (10): 1863-71.
Edilova M I, Akram A, Abdul-Sater A A. Innate immunity drives pathogenesis of rheumatoid arthritis. Biomed J. 2021; 44 (2): 172-182.
Elenkov I J, Iezzoni D G, Daly A, Harris A G. Cytokine dysregulation, inflammation, and well-being. Neuroimmunomodulation. 2005; 12 (5): 255-69.
Fulop T, Larbi A, Dupuis G, et al. Immunosenescence and inflamm-aging as two sides of the same coin: Friends or foes? Front Immunol. 2018; 8 (1960).
Gaekwad J, Zhang Y, Zhang W, Reeves J, Wolfert M A, Boons G J. Differential induction of innate immune responses by synthetic lipid A derivatives. J Biol Chem. 2010; 285 (38): 29375-86.
Gansevoort R T, Correa-Rotter R, Hemmelgarn B R, Jafar T H, Heerspink H J, Mann J F, et al. Chronic kidney disease and cardiovascular risk: epidemiology, mechanisms, and prevention. Lancet. (2013) 382:339-52. 10.1016/S0140-6736 (13) 60595-4
Guo J, Liu Z, Zhang D, et al. TLR4 agonist monophosphoryl lipid A alleviated radiation-induced intestinal injury. J Immunol Res. 2019; 2019:2121095.
Ismaili J, Rennesson J, Aksoy E, et al. Monophosphoryl lipid A activates both human dendritic cells and T cells. J Immunol. 2002; 168:926-32.
Jain A, Kaczanowska S, Davila E. IL-1 receptor-associated kinase signaling and its role in inflammation, cancer progression, and therapy resistance. Front Immunol. 2014; 5 (553).
Mata-Haro, Verónica et al. “The vaccine adjuvant monophosphoryl lipid A as a TRIF-biased agonist of TLR4.” Science (New York, N.Y.) vol. 316, 5831 (2007): 1628-32. doi: 10.1126/science.1138963.
Mian M F, Lauzon N M, Andrews D W, Lichty B D, Ashkar A A. FimH can directly activate human and murine natural killer cells via TLR4. Mol Ther. 2010; 18 (7): 1379-88.
Moura L I F, Silva L, Leal E C, Tellechea A, Cruz M T, Carvalho E. Neurotensin modulates the migratory inflammatory response of macrophages under hyperglycemic conditions. Biomed Res Int. 2013; 2013:941764.
Ng Q X, Soh A Y S, Loke W, Lim D Y, Yeo W S. The role of inflammation in irritable bowel syndrome (IBS). J Inflamm Res. 2018; 11:345-349. Published 2018 Sep. 21. doi: 10.2147/JIR.S174982
O'Connor G, Hart O M, Gardiner C M. Putting the natural killer cell in its place. Immunology. 2006; 117 (1): 1-10.
Owen A M, Fults J B, Patil N K, Hernandez A, Bohannon J K. TLR agonists as mediators of trained immunity: Mechanistic insight and immunotherapeutic potential to combat infection. Front Immunol. 2021; 11:622614.
Pifferi C, Fuentes R, Fernandez-Tejada A. Natural and synthetic carbohydrate-based vaccine adjuvants and their mechanisms of action. Nature Reviews Chemistry. 2021; 5:197-216.
Sharfuddin, A., Molitoris, B. Pathophysiology of ischemic acute kidney injury. Nat Rev Nephrol 7, 189-200 (2011). https://doi.org/10.1038/nrneph.2011.16
Thompson Journal of Leukocyte Biology Volume 78, December 2005 doi: 10.1189/jlb.0305172.
Watts B A, Tamayo E, Sherwood E R, Good D W. Monophosphoryl lipid A pretreatment suppresses sepsis- and LPS-induced proinflammatory cytokine production in the medullary thick ascending limb. Am J Physiol Renal Physiol. 2020; 319: F8-F18.
Wilson M D. Fibrogenesis: Mechanisms, Dynamics and Clinical Implications. Iran J Pathol. 2015; 10 (2): 83-88.
Xie Y, Bowe B, Mokdad A H, Xian H, Yan Y, Li T, et al., Analysis of the global burden of disease study highlights the global, regional, and national trends of chronic kidney disease epidemiology from 1990 to 2016. Kidney Int. (2018) 94:567-81. 10.1016/j.kint.2018.04.011
Zwirner Norberto Walter, Ziblat Andrea. Regulation of NK Cell Activation and Effector Functions by the IL-12 Family of Cytokines: The Case of IL-27. Frontiers in Immunology. VOL 8, 2017 https://www.frontiersin.org/article/10.3389/fimmu. 2017.00025 DOI=10.3389/fimmu.2017.00025

We claim:

1. A method for treating or preventing loss of organ function due to chronic organ diseases by administering to a subject in need thereof an effective amount of a toll-like receptor 4 (TLR4) agonist.

2. The method of claim 1 wherein the TLR4 agonist is an MPLA-like compound.

3. The method of claim 1 or 2 wherein the TLR4 is phosphorylated hexaacyl disaccharide (PHAD), 3-deacyl phosphorylated hexaacyl disaccharide (3D-PHAD), 3D-(6-acyl) phosphorylated hexaacyl disaccharide (3D(6-acyl) PHAD) or any combination thereof or pharmaceutically acceptable salts thereof.

4. The method of claim 3, wherein the TLR4 agonist is PHAD or a pharmaceutically acceptable salt thereof.

5. The method of any one of claims 2 to 4, wherein the MPLA-like compound is administered as an aqueous solution.

6. The method of any of claims 2 to 5 wherein the MPLA-like compound is delivered parenterally as an aqueous solution.

7. The method of any one of claims 2 to 4, wherein the MPLA-like compound is delivered orally as a tablet.

8. The method of any one of claims 1 to 7, wherein the TLR4 agonist selectively stimulates the TIR-domain-clustering adapter-inducing interferon-β (TRIF) pathway.

9. The method of any one of claims 1 to 8, wherein the chronic organ disease is selected from nonalcoholic fatty liver, nonalcoholic steatohepatitis, osteoarthritis, rheumatoid arthritis, irritable bowel syndrome, pulmonary fibrotic disease, heart disease, and any combination thereof.

10. The method of any one of claims 1 to 9, wherein the TLR4 agonist reduces circulating TGF-β in the subject.

11. The method of any one of claims 1 to 10, wherein TLR4 agonist increases circulating interleukin-10 (IL-10), circulating hepcidin and/or circulating neutrophil gelatinase-associated lipocalin (NGAL) in the subject.

12. The method of any one of claims 1 to 11, wherein the method prevents loss of organ function due to a chronic organ disease.

13. The method of any one of claims 1 to 12, wherein the method treats loss of organ function due to a chronic organ disease.

14. A method for treating or preventing loss of kidney function due to chronic kidney disease by administering to a subject in need thereof an effective amount of a toll-like receptor 4 (TLR4) agonist.

15. The method of claim 14 wherein the TLR4 agonist is an MPLA compound.

16. The method of claim 14 or 15, wherein the TLR4 agonist is phosphorylated hexaacyl disaccharide (PHAD), 3-deacyl phosphorylated hexaacyl disaccharide (3D-PHAD), 3D-(6-acyl) phosphorylated hexaacyl disaccharide (3D(6-acyl) PHAD) or any combination thereof or pharmaceutically acceptable salts thereof.

17. The method of claim 16, wherein the TLR4 agonist is PHAD or a pharmaceutically acceptable salt thereof.

18. The method of any one of claims 15 to 17, wherein the MPLA-like compound is administered as an aqueous solution.

19. The method of any one of claims 15 to 18, wherein the MPLA-like compound is delivered parenterally as an aqueous solution.

20. The method of any one of claims 15 to 17, wherein the MPLA-like compound is delivered orally as a tablet.

21. The method of any one of claims 14 to 20, wherein the loss of kidney function is associated with one or more of the following:
a. Diabetes Type I or Type II, hypertension, glomerulonephritis, polycystic kidney disease, autoimmune diseases, vesicoureteral reflux, pyelonephritis, interstitial nephritis, kidney stones, obstruction in kidney, cancer, substance abuse, kidney inflammation, kidney fibrosis, medication overuse, and chemotherapy.

22. The method of any one of claims 14 to 21, wherein the TLR4 agonist selectively stimulates the TIR-domain-clustering adapter-inducing interferon-β (TRIF) pathway.

23. The method of any one of claims 14 to 22, wherein the TLR4 agonist reduces circulating TGF-β in the subject.

24. The method of any one of claims 14 to 22, wherein the TLR4 agonist increases circulating interleukin-10 (IL-10), circulating hepcidin and/or circulating neutrophil gelatinase-associated lipocalin (NGAL) in the subject.

25. The method of any one of claims 14 to 24, wherein the method prevents loss of loss of kidney function due to chronic kidney disease

26. The method of any one of claims 14 to 25, wherein the method treats loss of loss of kidney function due to chronic kidney disease.

27. A method for treating or preventing loss of organ function due to acute stress by administering to a subject in need thereof an effective amount of a TLR4 agonist.

28. The method of claim 27, wherein the TLR4 agonist is an MPLA compound.

29. The method of claim 27 or 28, wherein the TLR4 agonist is phosphorylated hexaacyl disaccharide (PHAD), 3-deacyl phosphorylated hexaacyl disaccharide (3D-PHAD), 3D-(6-acyl) phosphorylated hexaacyl disaccharide (3D(6-acyl) PHAD) or any combination thereof or a pharmaceutically acceptable salts thereof.

30. The method of claim 29, wherein the TLR4 agonist is PHAD or a pharmaceutically acceptable thereof.

31. The method of any one of claims 28 to 30, wherein the MPLA-like compound is administered as an aqueous solution.

32. The method of any one of claims 28 to 31, wherein the MPLA-like compound is delivered parenterally as an aqueous solution.

33. The method of any one of claims 28 to 30, wherein the MPLA-like compound is delivered orally as a tablet.

34. The method of any one of claims 27 to 33, wherein the organ is a kidney.

35. The method of any one of claims 27 to 34, wherein the acute stress is due to one or more of the following:
a. toxicity from a medication, toxicity from chemotherapy, ischemia, trauma, cancer, or infection.

36. The method of any one of claims 27 to 35, wherein the TLR4 agonist selectively stimulates the TIR-domain-clustering adapter-inducing interferon-β (TRIF) pathway.

37. The method of any one of claims 27 to 36, wherein the TLR4 agonist reduces circulating TGF-β in the subject.

38. The method of any one of claims 27 to 37, wherein the TLR4 agonist increases circulating interleukin-10 (IL-10), circulating hepcidin and/or circulating neutrophil gelatinase-associated lipocalin (NGAL) in the subject.

39. The method of any one of claims 27 to 38, wherein the loss of organ function is due chronic inflammation or fibrosis.

40. The method of any one of claims 27 to 39, wherein the method prevents loss of organ function due to acute stress.

41. The method of any one of claims 27 to 40, wherein the method treats loss of organ function due to acute stress.

42. A pharmaceutical composition for treating or preventing loss of organ function in a subject in need thereof comprising a colloidal formulation of a monophosphoryl lipid A (MPLA)-like compound.

43. The pharmaceutical composition of claim 42, wherein the MPLA-like compound is selected from phosphorylated hexaacyl disaccharide (PHAD), PHAD-504, 3D-(6-acyl)-PHAD, 3D-PHAD, and any combination thereof.

44. The pharmaceutical composition of claim 43, wherein the MPLA-like compound is PHAD.

45. The pharmaceutical composition of any one of claims 43 to 45, wherein the pharmaceutical composition is an aqueous composition.

46. The pharmaceutical composition of any one of claims 42 to 45, wherein the pharmaceutical composition is a dry powder.

47. The pharmaceutical composition of claim 45, wherein the pharmaceutical composition has a MPLA concentration of about 1 μg/mL to about 10,000 μg/mL.

48. The pharmaceutical composition of any one of claims 42 to 47, further comprising a stabilizer.

49. The pharmaceutical composition of claim 48, wherein the stabilizer is trehalose.

50. The pharmaceutical composition of any of claims 42 to 49, wherein the composition comprises micelles having an average diameter or length of about 1 nm to about 1000 nm.

51. The pharmaceutical composition of any of claims 42 to 50, further comprising a bulking agent selected from one or more of the following: mannitol, trehalose, chitosan, HP-B-Cyclodextrin, hydroxypropylmethylcellulose (HPMC), dextran, pea starch, and sucrose.
